(19) United States
(12) Patent Application Publication
(10) Pub. No.: US 2025/0002904 A1
(43) Pub. Date: Jan. 2, 2025

(54) METHODS AND SYSTEMS FOR GENERATING SEQUENCING LIBRARIES

(71) Applicant: Adela, Inc., Foster City, CA (US)

(72) Inventors: Scott V. BRATMAN, Toronto (CA); Justin M. BURGENER, Toronto (CA); Rajat SINGHANIA, Toronto (CA); Shu Yi SHEN, Markham (CA); Iulia CIRLAN, Toronto (CA); Daniel DINIZ DE CARVALHO, Toronto (CA)

(21) Appl. No.: 18/735,441

(22) Filed: Jun. 6, 2024

Related U.S. Application Data
(63) Continuation of application No. PCT/US2022/052432, filed on Dec. 9, 2022.
(60) Provisional application No. 63/367,551, filed on Jul. 1, 2022.
(60) Provisional application No. 63/288,496, filed on Dec. 10, 2021.

(51) Int. Cl. C12N 15/10

(52) U.S. Cl. CPC C12N 15/1093

(57) ABSTRACT

Methods and systems for targeted detection of circulating tumor DNA (ctDNA) molecules are disclosed herein. In some cases, a molecular sequencing library depleted of methylated DNA can be generated and used to detect ctDNA in a cell-free DNA sample reliably at a lower sequencing depth and lower cost than existing methods.

### CROSS REFERENCE

This application is a continuation application of International Application No. PCT/US2022/052432, filed on Dec. 9, 2022, which claims the benefit of U.S. Provisional Patent Application No. 63/288,496, filed on Dec. 10, 2021, and U.S. Provisional Patent Application No. 63/367,551, filed on Jul. 1, 2022, which are each incorporated by reference in their entirety.

### BACKGROUND

Circulating tumor DNA (ctDNA) has increasingly demonstrated potential as a non-invasive, tumor-specific biomarker for routine clinical use. ctDNA is derived from tumor cells predominantly undergoing cell-death and released into circulation of various bodily fluids including blood. In most cancer patients, the majority of blood-derived cell-free DNA originates from healthy (e.g., non-cancerous) tissues. In addition, the fraction of ctDNA observed may range from <0.1% to 90% of total cell-free DNA at diagnosis depending on several factors including primary site of the tumor and disease burden. ctDNA has been providing non-invasive access to the tumor's molecular landscape and disease burden. Methods for detecting ctDNA with increased sensitivity are needed, especially in subjects with lower abundance of ctDNA.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In one aspect, the present disclosure provides a method for nucleic acid processing comprises: (a) providing a mixture comprising (i) a first plurality of nucleic acid molecules of a nucleic acid sample of a subject and (ii) a second plurality of nucleic acid molecules that is not from the subject, (b) contacting the mixture with a binder selective for methylated regions of nucleic acid molecules under a sufficient condition for the binder to bind the methylated regions of nucleic acid molecules, wherein the second plurality of nucleic acid molecules increases the binder's selectivity for a plurality of methylated regions of the first plurality of nucleic acid molecules; (c) with aid of the second plurality of nucleic acid molecules, depleting the mixture of one or more nucleic acid molecules of the first plurality of nucleic acid molecules having a methylation level at or above a threshold methylation level, thereby yielding a remainder of the first plurality of nucleic acid molecules having a methylation level below the threshold methylation level; and (d) identifying a sequence of the remainder of the first plurality of nucleic acid molecules.

In another aspect, the present disclosure provides a method for nucleic acid processing, wherein the method comprises: (a) providing a mixture comprising (i) a first plurality of nucleic acid molecules of a nucleic acid sample of a subject and (ii) a second plurality of nucleic acid molecules that is not from the subject; (b) with aid of the second plurality of nucleic acid molecules, depleting the mixture of one or more nucleic acid molecules of the first plurality of nucleic acid molecules that are hypermethylated, thereby yielding a remainder of the first plurality of nucleic acid molecules that is unmethylated or hypomethylated relative to the one or more nucleic acid molecules; and (c) identifying a sequence of the remainder of the first plurality of nucleic acid molecules. In some embodiments, a method further comprising contacting the mixture with a binder selective for methylated regions of nucleic acid molecules under a sufficient condition for the binder to bind the methylated regions of nucleic acid molecules. In some embodiments, the first plurality of nucleic acid molecules comprises deoxyribonucleic acid (DNA) molecules. In some embodiments, the nucleic acid sample is a cell-free DNA (cfDNA) sample.

In some embodiments, the second plurality of nucleic acid molecules comprises deoxyribonucleic acid (DNA) molecules. In some embodiments, the second plurality of nucleic acid molecules does not align to a human genome. In some embodiments, the second plurality of nucleic acid molecules is A DNA. In some embodiments, the second plurality of nucleic acid molecules comprises a fragment length of about 50 base pairs (bp) to about 800 bp. In some embodiments, the remainder of the first plurality of nucleic acid molecules comprises a fragment length of at least about 300 bp. In some embodiments, the remainder of the first plurality of nucleic acid molecules comprises a fragment length of at least about 100 bp to at least about 200 bp. In some embodiments, the remainder of the first plurality of nucleic acid molecules comprises a fragment length of at least about 120 bp to at least about 150 bp.

In some embodiments, the remainder of the first plurality of nucleic acid molecules is deprived of CpG genomic islands. In some embodiments, the remainder of the first plurality of nucleic acid molecules comprises long interspersed nuclear elements (LINEs). In some embodiments, the remainder of the first plurality of nucleic acid molecules comprises short interspersed nuclear elements (SINEs). In some embodiments, the remainder of the first plurality of nucleic acid molecules comprises long terminal repeat (LTR) elements. In some embodiments, the binder is selected from the group consisting of an anti-5-methylcytosine antibody or a derivative thereof, an anti-5-carboxylcytosine antibody or a derivative thereof, an anti-5-formylcytosine antibody or a derivative thereof, an anti-5-hydroxymethylcytosine antibody or a derivative thereof, an anti-3-methylcytosine antibody or a derivative thereof, and any combinations thereof. In some embodiments, the binder is the anti-5-methylcytosine antibody or a derivative thereof.

In some embodiments, a method (e.g., step (d)) comprises purifying the remainder of the first plurality of nucleic acid molecules to yield a plurality of purified nucleic acid molecules. In some embodiments, a method further comprises amplifying the plurality of purified nucleic acid molecules. In some embodiments, a method further comprises subjecting amplified nucleic acid molecules or derivative thereof to sequencing. In some embodiments, the sequencing is performed at a low sequencing depth. In some embodiments, the sequencing is performed at a sequencing depth of from 0.1× to 10×. In some embodiments, the sequencing is performed at a sequencing depth of from 0.1× to 5.0×. In some embodiments, the sequencing is performed at a sequencing depth of from 0.5× to 5.0×. In some embodiments, the sequencing is performed at a sequencing depth of from 0.5× to 10×.

In some embodiments, a method further comprises using an array or polymerase chain reaction (PCR) to identify a sequence of the first plurality of nucleic acid molecules or derivative thereof. In some embodiments, the remainder of the first plurality of nucleic acid molecules comprises a sum of Reads Per Kilobase per Million reads (RPKMs) that is lower than 50,000 across a plurality of CpG islands. In some embodiments, the remainder of the first plurality of nucleic acid molecules comprises a low sum of Reads Per Kilobase per Million reads (RPKMs) that is lower than 50,000 across a plurality of CpG island shores. In some embodiments, the remainder of the first plurality of nucleic acid molecules comprises a CpG enrichment score that is lower than 2.

In another aspect, the present disclosure provides a method for nucleic acid processing, comprises: (a) providing a nucleic acid sample comprising a plurality of nucleic acid molecules, wherein at least a portion of said plurality of nucleic acid molecules is circulating tumor nucleic acid molecules; (b) contacting said nucleic acid sample with a binder selective for methylated regions of nucleic acid molecules under a sufficient condition for the binder to bind the methylated regions of nucleic acid molecules; (c) depleting said plurality of nucleic acid molecules of one or more nucleic acid molecules that are hypermethylated, thereby yielding a remainder of said plurality of nucleic acid molecules that is unmethylated or hypomethylated relative to said one or more nucleic acid molecules, wherein said remainder of said plurality of nucleic acid molecules comprises said circulating tumor nucleic acid molecules; and (d) identifying a sequence of said remainder of said plurality of nucleic acid molecules or derivatives thereof.

In another aspect, the present disclosure provides a method for nucleic acid processing, comprising: (a) subjecting a plurality of nucleic acid molecules or derivatives thereof of a nucleic acid sample derived from a subject to sequencing to generate a plurality of sequencing reads, wherein the nucleic acid sample has been enriched for a hypomethylated or depleted for a hypermethylated region; (b) computer processing the plurality of sequencing reads to obtain a fragment length profile of the subject, wherein the fragment length profile comprises a first portion of the plurality of sequencing reads having a fragment length below a threshold fragment length and a second portion of the plurality of sequencing reads having a fragment length above the threshold fragment length; (c) using at least the fragment length profile to generate a fragment fraction score; and (d) using at least the fragment fraction score to determine whether the subject has or is at an increased risk of having a cancer.

In some embodiments, the method further comprises obtaining a first fraction of the first portion of sequencing reads and a second fraction of the second portion of sequencing reads. In some embodiments, the first fraction is obtained by dividing a first copy number of the first portion of sequencing reads by the first copy number plus a second copy number of the second portion of sequencing reads. In some embodiments, the second fraction is obtained by dividing the second copy number of the second portion of sequencing reads by the first copy number plus a second copy number of the second portion of sequencing reads. In some embodiments, the fragment fraction score comprises subtracting the second fraction from the first fraction. In some embodiments, the threshold fragment length is from about 140 bp to about 160 bp. In some embodiments, the threshold fragment length is about 150 bp. In some embodiments, the first portion of sequencing reads derived from nucleic acid molecules or derivatives thereof having a fragment length of about 100 bp to about 150 bp. In some embodiments, the first portion of sequencing reads derived from nucleic acid In some embodiments, the method further comprises to determining whether the subject has or is at an increased risk of having a cancer a specificity of at least about 90%. In some embodiments, the method further comprises to determining whether the subject has or is at an increased risk of having a cancer a specificity of at least about 95%. In some embodiments, the method further comprises to determining whether the subject has or is at an increased risk of having a cancer a specificity of at least about 98%. In some embodiments, the method further comprises administering a therapeutically effective dose of a treatment to the subject in need thereof, wherein the treatment is selected from the group consisting of surgery, chemotherapy, radiation therapy, targeted therapy, immunotherapy, cell therapy, an antihormonal agent, an antimetabolite chemotherapeutic agent, a kinase inhibitor, a methyltransferase inhibitor, a peptide, a gene therapy, a vaccine, a platinum-based chemotherapeutic agent, an antibody, a checkpoint inhibitor, and any combinations thereof. In some embodiments, a sequencing read of said sequencing reads is mappable to a specific region of a genome of said subject.

In another aspect, the present disclosure provides a method for nucleic acid processing, comprising: (a) subject a plurality of nucleic acid molecules or derivatives thereof of a nucleic acid sample derived from a subject to sequencing to a plurality of sequencing reads, wherein the sequencing is performed at a sequencing depth of from 0.1× to 10× and wherein the plurality of nucleic acid molecules or derivatives thereof comprises a methylation level at or below a threshold methylation level; (b) computer processing the plurality of sequencing reads to obtain a fragment length profile of the subject; (c) using at least the fragment length profile to generate a fragment fraction score; and (d) using at least the fragment fraction score to determine whether the subject has or is at an increased risk of having a cancer.

In some embodiments, the fragment length profile comprises a first portion of sequencing reads having a fragment length below a threshold fragment length and a second portion of sequencing reads having a fragment length above the threshold fragment length. In some embodiments, the method further comprises obtaining a first fraction of the first portion of sequencing reads and a second fraction of the second portion of sequencing reads. In some embodiments, the first fraction is obtained by dividing a first copy number of the first portion of sequencing reads by the first copy number plus a second copy number of the second portion of sequencing reads. In some embodiments, the second fraction is obtained by dividing the second copy number of the second portion of sequencing reads by the first copy number plus a second copy number of the second portion of sequencing reads. In some embodiments, obtaining the fragment fraction score comprises subtracting the second fraction from the first fraction. In some embodiments, wherein the threshold fragment length is from about 140 bp to about 160 bp. In some embodiments, the threshold fragment length is about 150 bp. In some embodiments, the first portion of sequencing reads derived from nucleic acid molecules or derivatives thereof having a fragment length of about 100 bp to about 150 bp. In some embodiments, the first portion of sequencing reads derived from nucleic acid molecules or derivatives thereof having a fragment length of about 151 bp to about 220 bp. In some embodiments, the method further comprises to determining whether the subject has or is at an increased risk of having a cancer a specificity of at least about 90%. In some embodiments, the method further comprises to determining whether the subject has or is at an increased risk of having a cancer a specificity of at least about 95%. In some embodiments, the method further comprises to determining whether the subject has or is at an increased risk of having a cancer a specificity of at least about 98%. In some embodiments, the method further comprises administering a therapeutically effective dose of a treatment to the subject in need thereof, wherein the treatment is selected from the group consisting of surgery, chemotherapy, radiation therapy, targeted therapy, immunotherapy, cell therapy, an antihormonal agent, an antimetabolite chemotherapeutic agent, a kinase inhibitor, a methyltransferase inhibitor, a peptide, a gene therapy, a vaccine, a platinum-based chemotherapeutic agent, an antibody, a checkpoint inhibitor, and any combinations thereof. In some embodiments, a sequencing read of the sequencing reads is mappable to a specific region of a genome of the subject.

In another aspect, the present disclosure provides a method for determining whether a subject has or is at an increased risk of having cancer, comprising: (a) obtaining a sample of the subject, wherein the sample comprises a plurality of nucleic acid molecules; (b) subjecting the plurality of nucleic acid molecules or a derivative thereof to sequencing to generate a plurality of sequencing reads; (c) computer processing the plurality of sequencing reads to generate a first fragment fraction score, wherein the first fragment fraction score is generated at least in part by: (i) determining a first number of the plurality of sequencing reads that have lengths between a first threshold and a second threshold greater than the first threshold; (ii) determining a second number of the plurality of sequencing reads that have lengths between the second threshold and a third threshold greater than the second threshold; (iii) generating the first fragment fraction score at least in part by (1) determining a difference between the first number and the second number, and (2) dividing the difference by a sum of the first number and the second number; (d) computer processing the first fragment fraction score generated in (c) against a second fragment fraction score generated from a healthy control to determine that the first fragmentation score is greater than the second fragmentation score; and (e) upon determining that the first fragment fraction score is greater than the second fragment fraction score, outputting a report that identifies the subject as having or being at an increased risk of having the cancer.

In some embodiments, a sequencing read of the sequencing reads is mappable to a specific region of a genome of the subject. In some embodiments, the plurality of nucleic acid molecules are hypomethylated. In some embodiments, the method further comprises, prior to (b), enriching the sample for the plurality of nucleic acid molecules that are hypomethylated; and the method further comprises, prior to (b), depleting the sample for nucleic acid molecules that are hypermethylated.

### BRIEF DESCRIPTION OF FIGURES

These and other features of the preferred embodiments of the invention will become more apparent in the following detailed description in which reference is made to the appended drawings wherein:

FIG. 1 shows a diagram illustrating a process for collecting flow-through of unmethylated/hypomethylated DNA fragments.

FIG. 2A shows sequencing counts observed from 5mC-enriched libraries derived from cfDNA samples following methylated DNA immunoprecipitation (MeDIP) pull-down with 5mC-specific binders, in accordance with embodiments of the present disclosure.

FIG. 2B shows sequencing counts observed from 5mC-depleted libraries derived from cfDNA samples following MeDIP pull-down with 5mC-specific binders, in accordance with embodiments of the present disclosure.

FIG. 3 shows a comparison of methylation specificity observed in 5mC-enriched and 5mC-depleted libraries derived from cfDNA samples, in accordance with embodiments of the present disclosure.

FIG. 4A shows methylated signal of the top 10% RPKM scoring 300-bp windows in CpG Islands of chromosome 1 for 5mC-enriched and 5mC-depleted libraries, in accordance with embodiments of the present disclosure.

FIG. 4B shows methylated signal of the top 10% RPKM scoring 300-bp windows in CpG Islands of chromosome 2 for 5mC-enriched and 5mC-depleted libraries, in accordance with embodiments of the present disclosure.

FIG. 4C shows methylated signal of the top 10% RPKM scoring 300-bp windows in CpG Islands of chromosome 3 for 5mC-enriched and 5mC-depleted libraries, in accordance with embodiments of the present disclosure.

FIG. 5A shows calculated CpG enrichments scores for 5mC-enriched libraries, in accordance with embodiments of the present disclosure.

FIG. 5B shows calculated CpG enrichments scores for 5mC-depleted libraries, in accordance with embodiments of the present disclosure.

FIG. 6A shows sums of RPKMs in CpG islands for 5mC-enriched libraries, in accordance with embodiments of the present disclosure.

FIG. 6B shows sums of RPKMs in CpG islands for 5mC-depleted libraries, in accordance with embodiments of the present disclosure.

FIG. 7A shows sums of RPKMs in CpG island shores for 5mC-enriched libraries, in accordance with embodiments of the present disclosure.

FIG. 7B shows sums of RPKMs in CpG island shores for 5mC-depleted libraries, in accordance with embodiments of the present disclosure.

FIG. 8A shows saturation analysis of cfMeDIP-seq data from each replicate for each input concentration of DNA mimic samples, in accordance with embodiments of the present disclosure.

FIG. 8B shows specificity of cfMeDIP-seq data for input DNA mimic concentrations of 100 ng, 10 ng, 5 ng, and 1 ng using methylated and unmethylated spike-in DNA (dotted line indicates fold-enrichment ratio threshold of 25; Error bars represent ±s.e.m.), in accordance with embodiments of the present disclosure.

FIG. 8C shows CpG enrichment scores for sequenced DNA mimic, in accordance with embodiments of the present disclosure.

FIG. 9A shows a schematic representation of serial dilution of colorectal cancer (CRC) DNA samples and multiple myeloma (MM) DNA samples, in accordance with embodiments of the present disclosure.

FIG. 9B shows specificity of reactions for each dilution of CRC DNA and MM DNA using methylated and unmethylated spike-in DNA, in accordance with embodiments of the present disclosure.

FIG. 9C shows CpG enrichment scores of CpGs within genomic regions from immunoprecipitated samples, in accordance with embodiments of the present disclosure.

FIG. 9D shows saturation analysis from dilutions of spike-in CRC DNA, in accordance with embodiments of the present disclosure.

FIG. 10 shows percent recovery of spike-in unmethylated DNA after cfMeDIP-seq, in accordance with embodiments of the present disclosure.

FIG. 11 shows percent recovery of spike-in methylated DNA after cfMeDIP-seq, in accordance with embodiments of the present disclosure.

FIG. 12 shows distributions of genome-wide Methylation Fraction Fragmentation (MFF) analysis, in accordance with embodiments of the present disclosure.

FIG. 13 shows distributions of Methylation Fraction Fragmentation (MFF) analysis limited to CpG shores, in accordance with embodiments of the present disclosure.

FIG. 14 shows distributions of Methylation Fraction Fragmentation (MFF) analysis limited to long terminal repeats (LTRs), in accordance with embodiments of the present disclosure.

FIG. 15 shows heatmap analysis of enriched MFFs of interest across enriched MFF libraries (MFFs), in accordance with embodiments of the present disclosure.

FIG. 16 shows PCA of enriched MFFs of interest, across all enriched MFF libraries, in accordance with embodiments of the present disclosure.

FIG. 17 shows heatmap analysis of depleted MFFs of interest, across all depleted MFF libraries, in accordance with embodiments of the present disclosure.

FIG. 18 shows PCA analysis of depleted MFFs of interest, across all depleted MFF libraries, in accordance with embodiments of the present disclosure.

FIG. 19 shows a heatmap of depleted MFFs of interest across all depleted MFF libraries and enriched MFFs of interest across all enriched MFF libraries, in accordance with embodiments of the present disclosure.

FIG. 20 shows a schematic of a computer system, in accordance with embodiments of the present disclosure.

### DETAILED DESCRIPTION

The present disclosure provides methods, systems, and kits for the processing and analysis of nucleic acids present in biological samples, which can be useful in determining a risk or likelihood of a subject having cancer or a tumor with high sensitivity, high specificity, or both. Methods, systems, and kits provided herein can comprise the creation, use, or both of nucleic acid libraries in determining the presence of circulating tumor DNA (ctDNA) in biological samples (e.g., biological samples comprising cell-free DNA, cfDNA), for example, to determine a subject's risk of having or developing a tumor or cancer. In particular, the present disclosure provides methods, systems, compositions, and kits for the creation and use of depleted sequencing libraries, which can allow for increased sensitivity, specificity, or both in determining the presence, sequence identity, or both of cancer-derived and/or tumor-derived nucleic acids in a biological sample. For instance, the provision or use of depleted sequencing libraries can allow for highly sensitive and highly specific detection and/or characterization of circulating tumor DNA (ctDNA) in a fluid sample (e.g., a blood sample) obtained from a subject. In some cases, the provision and/or use of depleted sequencing libraries (e.g., as disclosed herein) can allow for increased sensitivity, specificity, and/or efficiency in the determination of a subject's risk of having or having a risk of developing a tumor or cancer.

Cell-free DNA (cfDNA), which can be present in biological samples that can be collected non-invasively (e.g., blood, urine, saliva, cerebrospinal fluid (CSF), etc.), can be a heterogeneous population comprising both cfDNA derived from healthy tissues and cfDNA derived from tumor or cancer cells (e.g., ctDNA). Cancer development can be associated with focal gain of 5′ methylcytosines (5mC), for instance, at cytosine-phosphate-guanine (CpG) islands and CpG island shores. Cancer development can also be associated with global (e.g., genome-wide) cytosine demethylation (e.g., global loss of 5mC). In some cases, ctDNA can be distinguished from cfDNA molecules derived from healthy tissue (e.g., non-tumor and/or non-cancer tissue) by the methylation level (e.g., the percentage of nucleotide residues that are methylated) of the nucleic acid molecules. In some cases, nucleic acid molecules of or derived from tumor tissue and/or cancer tissue can be hypomethylated (e.g., can comprise a lower level of methylation, for instance, wherein there are fewer methylated nucleotide residues and/or a lower percentage of methylated nucleotide residues) compared to nucleic acid molecules of or derived from healthy tissue (e.g., nucleic acid molecules of or derived from healthy tissue that consist of or comprise nucleotide sequences corresponding to the same region(s) of the genome of the subject). For example, tumor-derived nucleic acid molecules (e.g., ctDNA molecules) can comprise one or more regions having fewer methylated nucleotide residues than nucleic acid molecules (e.g., cfDNA molecules) derived from healthy tissues (e.g., non-tumor and/or non-cancer tissues) in the same biological sample. In some cases, all or a portion of a tumor-derived fraction of a plurality of cell-free DNA molecules (e.g., ctDNA) can be distinguished from cfDNA molecules derived from healthy tissue by one or more biophysical properties (e.g., the length of the cfDNA molecules or the presence of stereotypical 5′ and 3′ end sequence motifs) and/or one or more fragmentomics patterns. For instance, ctDNA molecules can have shorter nucleic acid lengths than cfDNA molecules derived from healthy tissues. In some cases, ctDNA molecules may comprise stereotypical 5′ and 3′ end motifs. In some cases, one or more of these distinguishing features may be used to deplete a population of nucleic acid molecules of cfDNA derived from healthy tissue and/or to enrich a population of nucleic acid molecules for ctDNA. ctDNA typically has shorter fragment length compared to cfDNA derived from a healthy tissue.

Nucleic acid molecules derived from tumor or cancer cells or tissue (e.g., ctDNA) may be present in a biological sample (and/or a population of nucleic acids derived from the biological sample) in substantially lower quantities than nucleic acid molecules (e.g., cfDNA) derived from healthy tissue. It can be difficult to detect or sequence (e.g., determine a sequence identity of) ctDNA present in a plurality of nucleic acid molecules (e.g., cfDNA) in or derived from a biological sample, for instance, because they are present in the sample in lower quantities relative to cfDNA derived from healthy tissue (e.g., which may require using a greater amount of potentially scarce biological sample and/or which may require significantly higher sequencing depth, if it is possible at all).

Depletion (e.g., removal) of all or a portion of the population of methylated DNA molecules (e.g., molecules having increased nucleotide methylation levels throughout or in a subset of the regions of the genome represented by the plurality of nucleic acid molecules of a biological sample) from a plurality of nucleic acid molecules (e.g., a plurality of cell-free nucleic acid molecules, or amplicons thereof, comprising a biological sample) may yield a remainder population of the plurality of nucleic acids of the biological sample that may be useful for determining a presence and/or sequence identity of ctDNA molecules in the biological sample. Typically, depletion/removing may be performed by using a binder specific for methylated DNA molecules to pull them down. The pull-down is typically collected and the flow-through containing the unmethylated/hypomethylated DNA molecules is discarded. The current disclosure provides for the first time methods and systems to collect such flow-through containing unmethylated/hypomethylated DNA molecules and to generate sequencing library using methylated/hypomethylated DNA molecules or derivatives thereof.

In some cases, a depleted sequencing library of methods, systems, compositions, and kits disclosed herein may consist of or can be comprised of such a remainder population of nucleic acid molecules. In some cases, it may be sufficient to deplete a plurality of nucleic acids (e.g., cfDNA molecules or amplicons thereof derived from a biological sample) of nucleic acid molecules methylated in one or more specific regions of the genomic sequence of the nucleic acid molecules (e.g., CpG islands, CpG island shores, or repetitive sequences of the genome, such as long interspersed nuclear elements (LINEs), short interspersed nuclear elements (SINEs), or LTRs (long terminal repeats)) to achieve increased sensitivity and/or increased specificity in assays for determining the presence or absence or the sequence identity of ctDNA molecules in the plurality. In some cases, a plurality of nucleic acids (e.g., cfDNA molecules or amplicons thereof derived from a biological sample) may be subjected to genome-wide depletion of nucleic acid molecules methylated in one or more specific regions of the genomic sequence of the nucleic acid molecules (e.g., CpG islands, CpG island shores, or repetitive sequences of the genome, such as long interspersed nuclear elements (LINEs), short interspersed nuclear elements (SINEs), or LTRs (long terminal repeats)) to achieve increased sensitivity and/or increased specificity in assays for determining the presence or absence or the sequence identity of ctDNA molecules in the plurality. In some cases, a remainder population (e.g., a plurality of nucleic acid fragments useful in the creation of a depleted library) can be deprived of CpG genomic islands. In some cases, a remainder population (e.g., a plurality of nucleic acid fragments useful in the creation of a depleted library) can comprise one or more of: long interspersed nuclear elements (LINEs), short interspersed nuclear elements (SINEs), or long terminal repeat (LTR) elements.

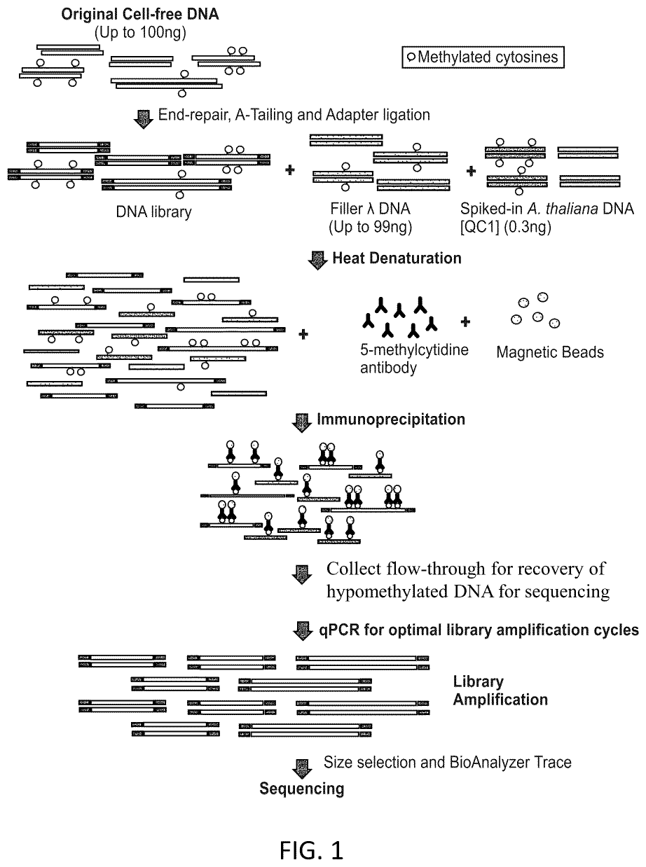

Depletion of all or a portion of the methylated nucleic acid molecules of a plurality of nucleic acid molecules of a biological sample may comprise contacting the methylated nucleic acid molecules with a binder (e.g., an affinity molecule, such as an antibody or a protein, specific to methylated nucleotide residues). For example, creation of a depleted sequencing library can comprise contacting a plurality of nucleic acid molecules (e.g., cfDNA molecules) or amplicons thereof with a binder selective for a methylated region of nucleic acid molecules (e.g., a methylcytosine binder (MBD), such as an MBD-Fc fusion protein). In some cases, a binder may be specific to one or more methylated nucleotide species (e.g., 5-methylcytosine (5mC)), for instance, as shown in FIG. 1. Cell-free Methylated DNA Immunoprecipitation sequencing (cfMeDIP-seq), a genome-wide molecular profiling technique, can enrich for methylated cfDNA fragments through use of a binder, such as an anti-5-methylcytosine (anti-5mC) antibody or methyl-CpG-binding domain (MBD) protein (e.g., MBD-Fc fusion proteins). As described herein, cfMeDIP-seq can comprise a portion of methods and systems for depleting a cfDNA sample of methylated DNA fragments, leaving behind hypomethylated or unmethylated cfDNA fragments, such as ctDNA. Thus, the identification of hypomethylated or unmethylated cell-free DNA within a clinical sample may be useful in determining the presence of a tumor or cancer in a subject.

In some cases, depletion of a plurality of nucleic acid molecules (e.g., in the creation of a depleted sequencing library and/or the determination of a presence or sequence identity of a nucleic acid molecule) may comprise removing one or more nucleic acid molecules having a methylation level above a threshold methylation level (e.g., wherein the one or more removed nucleic acid molecules are hypermethylated, for instance, relative to one or more nucleic acid molecules not removed during depletion). In some cases, a methylation level of a particular nucleic acid fragments (e.g., DNA fragments) may be considered to reach the threshold methylation level when a binder with a sufficient specificity for methylated cytosines is able to bind to the particular nucleic acid fragments either with or without using filler DNA as described here. In some cases, a methylation level of particular nucleic acid fragments (e.g., DNA fragments) may be considered to be below the threshold methylation level when a binder with a sufficient specificity for methylated cytosines is not able to bind to the particular nucleic acid fragments either with or without using filler DNA as described here. In some cases, depletion of a plurality of nucleic acid molecules (e.g., in the creation of a depleted sequencing library and/or the determination of a presence or sequence of a nucleic acid molecule) results in (e.g., provides) a remainder population of the plurality of nucleic acid molecules, wherein the remainder of the plurality of nucleic acid molecules comprises (or, in some cases, consists of) nucleic acid molecules having a methylation level below the threshold methylation level (e.g., wherein the remainder population is hypomethylated/unmethylated relative to one or more nucleic acid molecules removed from the plurality of nucleic acid molecules during depletion). A methylation level may be calculated as a percentage of hypermethylated nucleic acid fragments compared to all the nucleic acid fragments contained in a sample. In some cases, a threshold methylation level can be from 0.1% to 1%, 1% to 5%, 5% to 10%, 10% to 15%, 15% to 20%, 20% to 25%, 25% to 30%, 30% to 35%, 35% to 40%, 40% to 45%, 45% to 50%, 50% to 55%, 55% to 60%, 65% to 70%, 70% to 75%, 75% to 80%, 80% to 85%, 85% to 90%, 95% to 100%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at most 1%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%.

In some cases, a first plurality of nucleic acid molecules (e.g., comprising nucleic acid molecules, such as cfDNA, from a biological sample of a subject) may be combined (e.g., mixed) with a second plurality of nucleic acid molecules (e.g., wherein the second plurality of nucleic acid molecules is not from the subject from whom the biological sample was taken), for instance, as shown in FIG. 1. In some cases, the second plurality of nucleic acid molecules comprises supplemental processed DNA (e.g., comprising λ DNA). In some cases, each of the second plurality of nucleic acid molecules does not align to a human genome.

In some cases, a method or system disclosed herein may comprise determining or identifying a sequence of all or a portion of a depleted nucleic acid molecule population (e.g., remainder population of a plurality of nucleic acid fragments of a biological sample after pulling down hypermethylated nucleic acid fragments), for example, using a sequencer (e.g., as shown in FIG. 1). In some cases, a remainder population of nucleic acid molecules may be purified (e.g., after library creation) to yield a plurality of purified nucleic acid molecules, for example, prior to or as part of a process of determining or identifying a sequence of all or a portion of the depleted nucleic acid molecule population. In some cases, all or a portion of the plurality of purified nucleic acid molecules may be amplified (e.g., via polymerase chain reaction), for instance, prior to or as part of a process of determining or identifying a sequence of all or a portion of the depleted nucleic acid molecule population. In some cases, a population of amplified nucleic acid molecules or a derivative thereof (e.g., comprising amplicons of all or a portion of the plurality of purified nucleic acid molecules) may be subjected to sequencing (e.g., for the determination and/or identification of a sequence of the nucleic acid molecules). In some cases, the sequencing may be achieved using a sequencer, as described herein. In some cases, a sequence of a plurality of nucleic acid molecules of a biological sample (or a derivative thereof) may be identified or determined using an array or polymerase chain reaction. In some cases, the presence of a tumor-derived nucleic acid molecule may be determined by calculating a sum of reads per kilobase per million (RPKM) for a region of the genome (e.g., all or a portion of the genome, such as just CpG islands or just CpG island shores). In some cases, the presence of a tumor-derived nucleic acid molecule may be indicated when a depleted sequencing library (e.g., comprising a remainder population of nucleic acids) is observed to have a low sum of RPKMs, e.g., lower than 70,000, lower than 60,000, lower than 50,000, lower than 40,000, or lower than 30,000 across one or more regions of interest (e.g., CpG islands or CpG island shores).

### Supplemental Processed DNA (Filler DNA)

In some cases, supplemental processed DNA (e.g., filler DNA) may be added to a first plurality of nucleic acids (e.g., a plurality of nucleic acids from a biological sample, which may comprise cfDNA from healthy tissue and/or cfDNA from tumor tissue, such as ctDNA), for instance as shown in FIG. 1. In some cases, addition of supplemental processed DNA (e.g., a second plurality of nucleic acid molecules) to a first plurality of nucleic acid molecules can increase the specificity and/or sensitivity of a method, system, or kit described herein, for instance, with respect to the detection and/or identification of a nucleic acid sequence of the first plurality of nucleic acid molecules. In some cases, addition of supplemental processed DNA (e.g., a second plurality of nucleic acid molecules) to a first plurality of nucleic acid molecules may increase the rate of depletion of a methylated region of a nucleic acid sequence, e.g., during the practice of some embodiments of methods and systems described herein. In some cases, addition of supplemental processed DNA (e.g., a second plurality of nucleic acid molecules) to a first plurality of nucleic acid molecules (e.g., comprising cfDNA of a biological sample) may increase a binder's selectivity for one or more (e.g., a plurality of) methylated regions of the first plurality of nucleic acid molecules. In some cases, supplemental processed DNA (e.g., the second plurality of nucleic acid molecules) may be added to the first plurality of nucleic acid molecules in an amount sufficient to bring the combined mixture of nucleic acid molecules to a desired total mass. In some cases, a desired total mass for use in a method or system described herein can be from 20 ng to 30 ng, from 30 ng to 40 ng, from 40 ng to 50 ng, from 50 ng to 60 ng, from 60 ng to 70 ng, from 70 ng to 80 ng, from 80 ng to 90 ng, from 90 ng to 100 ng, from 100 ng to 110 ng, from 110 ng to 120 ng, from 120 ng to 130 ng, from 130 ng to 140 ng, from 140 ng to 150 ng, from 150 ng to 160 ng, from 160 ng to 170 ng, from 170 ng to 180 ng, from 180 ng to 190 ng, from 190 ng to 200 ng, greater than 200 ng, or less than 20 ng. In some cases, an amount of supplemental processed DNA from 1 ng to 5 ng, from 5 ng to 10 ng, from 10 ng to 20 ng, from 20 ng to 30 ng, from 30 ng to 40 ng, from 40 ng to 50 ng, from 50 ng to 60 ng, from 60 ng to 70 ng, from 70 ng to 80 ng, from 80 ng to 90 ng, from 90 ng to 100 ng, from 100 ng to 110 ng, from 110 ng to 120 ng, from 120 ng to 130 ng, from 130 ng to 140 ng, from 140 ng to 150 ng, from 150 ng to 160 ng, from 160 ng to 170 ng, from 170 ng to 180 ng, from 180 ng to 190 ng, from 190 ng to 200 ng, greater than 200 ng, less than 20 ng, less than 10 ng, or less than 5 ng can be added to a first plurality of nucleic acid molecules (e.g., to bring the total mixture of the supplemental processed DNA and the first plurality of nucleic acid molecules to the desired total mass). In some embodiments, the present disclosure comprises methods and systems for filling in the sample with an amount of supplemental processed DNA (e.g., filler DNA) to generate a mixture sample, wherein the mixture sample comprises at least about 50 ng, 55 ng, 60 ng, 65 ng, 70 ng, 75 ng, 80 ng, 85 ng, 90 ng, 95 ng, 100 ng, 120 ng, 140 ng, 160 ng, 180 ng, 200 ng, or any amount in between the numbers of the total amount of the nucleic acid mixture. In some embodiments, the supplemental processed DNA comprises at least about 5%, 10%, 15%, 20%, 30%, 40%, 50%, 60%, 70%, 80%, 90%, or 100% methylated supplemental processed DNA with remainder being unmethylated supplemental processed DNA, and in some cases between 5% and 50%, between 10%-40%, or between 15%-30% methylated supplemental processed DNA. In some embodiments, the mixture sample comprise an amount of supplemental processed DNA from 20 ng to 100 ng, in some cases 30 ng to 100 ng, in some cases 50 ng to 100 ng. In some embodiments, the cell-free DNA from the sample and the first amount of supplemental processed DNA together comprises at least 50 ng of total DNA, in some cases at least 100 ng of total DNA.

In some cases, supplemental processed DNA may be produced by fragmentation (e.g., via sonication). In some embodiments, the supplemental processed DNA may be 50 bp to 800 bp long, in some cases 100 bp to 600 bp long, and in some cases 200 bp to 600 bp long. In some embodiments, the supplemental processed DNA is double stranded. The supplemental processed DNA may be double stranded DNA. For example, the supplemental processed DNA may be junk DNA. The supplemental processed DNA may also be endogenous or exogenous DNA. For example, the supplemental processed DNA may be non-human DNA, and in some cases, A DNA. As used herein, “A DNA” generally refers to Enterobacteria phage A DNA. In some embodiments, the supplemental processed DNA has substantially no alignment to human DNA.

### Samples

A sample can be any biological sample isolated from a subject. For example, a sample may comprise, without limitation, bodily fluid, whole blood, platelets, serum, plasma, stool, white blood cells or leukocytes, endothelial cells, tissue biopsies, synovial fluid, lymphatic fluid, ascites fluid, interstitial or extracellular fluid, the fluid in spaces between cells, including gingival crevicular fluid, bone marrow, cerebrospinal fluid, saliva, mucous, sputum, semen, sweat, urine, fluid from nasal brushings, fluid from a pap smear, or any other bodily fluids. A bodily fluid may include saliva, blood, or serum. A sample may also be a tumor sample, which may be obtained from a subject by various approaches, including, but not limited to, venipuncture, excretion, ejaculation, massage, biopsy, needle aspirate, lavage, scraping, surgical incision, or intervention or other approaches. A sample may be a cell-free sample (e.g., substantially free of cells). DNA samples may be denatured, for example, using sufficient heat.

The sample may be taken from a subject with a disease or disorder. The sample may be taken from a subject suspected of having a disease or a disorder. In some embodiments, the sample may be obtained before and/or after treatment of a subject with a disease or disorder. Samples may be obtained from a subject during a treatment or a treatment regime. Multiple samples may be obtained from a subject to monitor the effects of the treatment over time. The disease or disorder may be a cancer. Specific examples of cancer types include suitable for detection with the methods according to the disclosure include acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilm's tumor. In an embodiment, the cancer is head and neck squamous cell carcinoma.

The sample may be taken from a healthy individual. In some cases, samples may be taken longitudinally from the same individual. In some cases, samples acquired longitudinally may be analyzed with the goal of monitoring individual health and early detection of health issues. In some embodiments, the sample may be collected at a home setting or at a point-of-care setting and subsequently transported by a mail delivery, courier delivery, or other transport method prior to analysis. For example, a home user may collect a blood spot sample through a finger prick, which blood spot sample may be dried and subsequently transported by mail delivery prior to analysis. In some cases, samples acquired longitudinally may be used to monitor response to stimuli expected to impact healthy, athletic performance, or cognitive performance. Non-limiting examples include response to medication, dieting, or an exercise regimen.

In some embodiments, the present disclosure provides a system, method, or kit that includes or uses one or more biological samples. The one or more samples used herein may comprise any substance containing or presumed to contain nucleic acids. A sample may include a biological sample obtained from a subject. In some embodiments, a biological sample is a liquid sample.

In some embodiments, the sample comprises less than about 100 ng, 90 ng, 80 ng, 75 ng, 70 ng, 60 ng, 50 ng, 40 ng, 30 ng, 20 ng, 10 ng, 5 ng, 1 ng or any amount in between the numbers of cell-free nucleic acid molecules. Further, in some embodiments, the sample comprises less than about 1 pg, less than about 5 pg, less than about 10 pg, less than about 20 pg, less than about 30 pg, less than about 40 pg, less than about 50 pg, less than about 100 pg, less than about 200 pg, less than about 500 pg, less than about 1 ng, less than about 5 ng, less than about 10 ng, less than about 20 ng, less than about 30 ng, less than about 40 ng, less than about 50 ng, less than about 100 ng, less than about 200 ng, less than about 500 ng, less than about 1000 ng, or any amount in between the numbers of cell-free nucleic acid molecules.

In some cases, creation or provision of a plurality of nucleic acid molecules from a biological sample can comprise performing one or more of end-repair, A-tailing, and adapter ligation on the plurality of nucleic acid molecules (e.g., after purification from the biological sample).

In some embodiments, a sample may be taken at a first time point and sequenced, and then another sample may be taken at a subsequent time point and sequenced. Such methods may be used, for example, for longitudinal monitoring purposes to track the development or progression of a disease. In some embodiments, the progression of a disease may be tracked before treatment, after treatment, or during the course of treatment, to determine the treatment's effectiveness. For example, a method as described herein may be performed on a subject prior to, and after, a medical treatment to measure the disease's progression or regression in response to the medical treatment.

After obtaining a sample from the subject, the sample may be processed to generate datasets indicative of a disease or disorder of the subject. For example, a presence, absence, or quantitative assessment of cell-free nucleic acid molecules (e.g., ctDNA molecules) of the sample at a panel of cancer-associated genomic loci or microbiome-associated loci may be indicative of a cancer of the subject. Processing the sample obtained from the subject may comprise (i) subjecting the sample to conditions that are sufficient to isolate, enrich, or extract a plurality of cell-free nucleic acid molecules, and (ii) assaying the plurality of cell-free nucleic acid molecules to generate the dataset (e.g., nucleic acid sequences). In some embodiments, a plurality of cell-free nucleic acid molecules is extracted from the sample and subjected to sequencing to generate a plurality of sequencing reads.

In some embodiments, the cell-free nucleic acid molecules may comprise cell-free ribonucleic acid (cfRNA) or cell-free deoxyribonucleic acid (cfDNA). The cell-free nucleic acid molecules (e.g., cfRNA or cfDNA) may be extracted from the sample by a variety of methods. The cell-free nucleic acid molecule may be enriched by a plurality of probes configured to enrich nucleic acid (e.g., RNA or DNA) molecules corresponding to a panel of cancer-associated genomic loci. The probes may have sequence complementarity with nucleic acid sequences from one or more of the panel of cancer-associated genomic loci. The panel of cancer-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct cancer-associated genomic loci. The probes may be nucleic acid molecules (e.g., RNA or DNA) having sequence complementarity with nucleic acid sequences (e.g., RNA or DNA) of the one or more genomic loci (e.g., cancer-associated genomic loci). These nucleic acid molecules may be primers or enrichment sequences. The assaying of the sample using probes that are selective for the one or more genomic loci (e.g., cancer-associated genomic loci or microbiome-associated loci) may comprise use of array hybridization, polymerase chain reaction (PCR), or nucleic acid sequencing (e.g., RNA sequencing or DNA sequencing).

Certain methods of capturing cell-free methylated DNA are described in WO 2017/190215 and WO 2019/010564, both of which are incorporated by reference in their entireties and for all purposes.

### Methylation Depleted Sequencing Libraries

Sequencing libraries depleted of methylated nucleic acids (e.g., a “depleted library” or a “methylation depleted library”) may improve the specificity, the sensitivity, and/or the efficiency of methods, systems, and kits for processing nucleic acids. For example, sequencing libraries depleted of methylated nucleic acids may improve the specificity, the sensitivity, and/or the efficiency of assays for determining the presence and/or sequence identity of a nucleic acid sequence. A sequencing library depleted of methylated nucleic acids may comprise a plurality of nucleic acids and/or fragments thereof. In some cases, a sequencing library depleted of methylated nucleic acids (e.g., a “depleted library” or “methylation depleted library”) may comprise a plurality of nucleic acid molecules (e.g., a population of nucleic acids and/or fragments thereof). The plurality of nucleic acid molecules may comprise all or a portion of a first plurality of nucleic acid molecules, e.g., wherein the first plurality of nucleic acid molecules comprises one or more nucleic acid molecules that comprise a methylated nucleic acid residue and one or more nucleic acid molecules that does not comprise a methylated nucleic acid residue. In some cases, a methylated nucleic acid may comprise one or more methylated nucleic acid residues. For instance, a methylated nucleic acid may comprise one or more methylated cytosines (e.g., one or more 5-methylcytosines (5mC) and/or one or more 5-hydroxymethylcytosines (5hmC)). A plurality of nucleic acid molecules (e.g., a plurality of nucleic acid molecules derived from a biological sample) may be depleted of methylated nucleic acid molecules by using a binder, e.g., as described herein, to form a depleted sequencing library. In some cases, a first plurality of nucleic acid molecules (e.g., comprising a plurality of cfDNA molecules derived from a biological sample) may be mixed with a second plurality of nucleic acid molecules (e.g., comprising supplemental processed DNA) before use of a binder to create a depleted sequencing library. In some cases, a sequencing library depleted of methylated nucleic acids may be fully depleted of methylated nucleic acid molecules. For instance, a sequencing library can comprise no (0%) methylated nucleic acid residues (e.g., a sequencing library containing no methylated cytosine residues). In some cases, a sequencing library depleted of methylated nucleic acids may be partially depleted of methylated nucleic acid molecules. In some cases, a sequencing library depleted of methylated nucleic acids may be depleted of nucleic acids having methylated nucleotides in one or more specific regions of a genomic sequence (e.g., CpG islands or CpG island shores).

### Nucleic Acid Molecule Sequencing

The present disclosure provides methods and technologies for determining the sequence of nucleotide bases in one or more polynucleotides. The polynucleotides may be, for example, nucleic acid molecules such as deoxyribonucleic acid (DNA) or ribonucleic acid (RNA), including variants or derivatives thereof (e.g., single stranded DNA). Sequencing may be performed by various systems currently available, such as, without limitation, a sequencing system by Illumina®, Pacific Biosciences (PacBio®), Oxford Nanopore®, or Life Technologies (Ion Torrent®). Further, any sequencing methods that provide fragment length such as paired-end sequencing may be utilized. Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

In some embodiments, the sequencing reads are obtained via a next-generation sequencing method or a next-next-generation sequencing method. In some embodiments, the sequencing methods comprise cfMeDIP sequencing, e.g., comprising processes or systems as described by Shen et al., (“Sensitive tumor detection and classification using plasma cell-free DNA methylomes,” (2018) Nature), which is incorporated herein in its entirety. In some embodiments, sequencing can be performed using methyl-CpG-binding domain sequencing (MBD-seq). In some cases, MBD-seq can comprise capture (e.g., via a binder, such as an antibody specific to a species of methylated nucleotide) of double-stranded, methylated DNA fragments for sequencing of methylation-enriched DNA fragment libraries. In some embodiments, the sequencing methods comprises CAncer Personalized Profiling by deep Sequencing (CAPP-Seq), which is a next-generation sequencing based method used to quantify circulating DNA in cancer (ctDNA). This method may be generalized for any cancer type that is documented to have recurrent mutations and may detect one molecule of mutant DNA in 10,000 molecules of healthy DNA. In some embodiments, the sequencing comprises bisulfite sequencing. In some embodiments, the sequencing does not comprise bisulfite sequencing.

In some cases, a sample or portion thereof (e.g., a plurality of nucleic acids of a sample) may be subjected to library preparation before sequencing. In short, after end-repair and A-tailing, the samples are ligated to nucleic acid adapters and digested using enzymes.

In some embodiments, sequencing comprises modification of a nucleic acid molecule or fragment thereof, for example, by ligating a barcode, a unique molecular identifier (UMI), or another tag to the nucleic acid molecule or fragment thereof. Ligating a barcode, UMI, or tag to one end of a nucleic acid molecule or fragment thereof may facilitate analysis of the nucleic acid molecule or fragment thereof following sequencing. In some embodiments, a barcode is a unique barcode (e.g., a UMI). In some embodiments, a barcode is non-unique, and barcode sequences may be used in connection with endogenous sequence information such as the start and stop sequences of a target nucleic acid (e.g., the target nucleic acid is flanked by the barcode and the barcode sequences, in connection with the sequences at the beginning and end of the target nucleic acid, creates a uniquely tagged molecule). A barcode, UMI, or tag may be a known sequence used to associate a polynucleotide or fragment thereof with an input or target nucleic acid molecule or fragment thereof. A barcode, UMI, or tag may comprise natural nucleotides or non-natural (e.g., modified) nucleotides (e.g., as described herein). A barcode sequence may be contained within an adapter sequence such that the barcode sequence may be contained within a sequencing read. A barcode sequence may comprise at least 4, 5, 6, 7, 8, 9, 10, 11, 12, 13, 14, 15, 16, or more nucleotides in length. In some cases, a barcode sequence may be of sufficient length and may be sufficiently different from another barcode sequence to allow the identification of a sample based on a barcode sequence with which it is associated. A barcode sequence, or a combination of barcode sequences, may be used to tag and subsequently identify an “original” nucleic acid molecule or fragment thereof (e.g., a nucleic acid molecule or fragment thereof present in a sample from a subject). In some cases, a barcode sequence, or a combination of barcode sequences, is used in conjunction with endogenous sequence information to identify an original nucleic acid molecule or fragment thereof. For example, a barcode sequence, or a combination of barcode sequences, may be used with endogenous sequences adjacent to a barcode, UMI, or tag (e.g., the beginning and end of the endogenous sequences).

As described herein, the prepared libraries may be combined with filler nucleic acids (e.g., filler A DNAs) to minimize the effect of low abundance ctDNA in the prepared libraries and generate mixed samples. In some embodiments, when the disease/condition is a locoregional (non-metastatic) cancer, the amount of ctDNA can be low and may not be easily and accurately measured and quantified. In such cases, the mixed samples may be brought to at least about 50 ng, 80 ng, 100 ng, 120 ng, 150 ng, or 200 ng and are subjected to further enrichment.

Processing a nucleic acid molecule or fragment thereof may comprise performing nucleic acid amplification. For example, any type of nucleic acid amplification reaction may be used to amplify a target nucleic acid molecule or fragment thereof and generate an amplified product. Non-limiting examples of nucleic acid amplification methods include reverse transcription, primer extension, polymerase chain reaction (PCR), ligase chain reaction, asymmetric amplification, rolling circle amplification, and multiple displacement amplification (MDA). Examples of PCR include, but are not limited to, quantitative PCR, real-time PCR, digital PCR, emulsion PCR, hot start PCR, multiplex PCR, asymmetric PCR, nested PCR, and assembly PCR. Nucleic acid amplification may involve one or more reagents such as one or more primers, probes, polymerases, buffers, enzymes, and deoxyribonucleotides. Nucleic acid amplification may be isothermal or may comprise thermal cycling. and/or with the length of the endogenous sequence.

### Binders

A binder may be used to deplete a population of nucleic acid molecules (e.g., a plurality of nucleic acid molecules derived from a biological sample). In some cases, a binder can be used to deplete a plurality of nucleic acid molecules of one or more nucleic acid molecules having a methylation level at or above a threshold methylation level (e.g., by binding to one or more methylated nucleotides of the one or more nucleic acid molecules). A binder may be used to enrich a population of nucleic acid molecules (e.g., a plurality of nucleic acids derived from a biological sample). In some cases, a binder can be specific to one or more methylated nucleotide species (e.g., 5-methylcytosine (5mC), 5-hydroxymethylcytosine (5hmC), 4-methylcytosine (4mC), or 6-methyladenine (6mA)). In some cases, a binder can be selected from the group consisting of an anti-5-methylcytosine antibody or a derivative thereof, an anti-5-carboxylcytosine antibody or a derivative thereof, an anti-5-formylcytosine antibody or a derivative thereof, an anti-5-hydroxymethylcytosine antibody or a derivative thereof, an anti-3-methylcytosine antibody or a derivative thereof, and any combinations thereof. In some cases, the binder can be an anti-5-methylcytosine antibody or a derivative thereof. In some embodiments, the binder is a protein comprising a Methyl-CpG-binding domain. One such protein is MBD2 protein. As used herein, “Methyl-CpG-binding domain (MBD)” generally refers to certain domains of proteins and enzymes that are approximately 70 residues long and bind to DNA that contains one or more symmetrically methylated CpGs. The MBD of MeCP2, MBD1, MBD2, MBD4 and BAZ2 mediates binding to DNA, and in cases of MeCP2, MBD1 and MBD2, preferentially to methylated CpG. Human proteins MECP2, MBD1, MBD2, MBD3, and MBD4 comprise a family of nuclear proteins related by the presence in each of a methyl-CpG-binding domain (MBD). Each of these proteins, with the exception of MBD3, is capable of binding specifically to methylated DNA.

In other embodiments, the binder is an antibody and capturing cell-free methylated DNA comprises immunoprecipitating the cell-free methylated DNA using the antibody. As used herein, “immunoprecipitation” generally refers a technique of precipitating an antigen (such as polypeptides and nucleotides) out of solution using an antibody that specifically binds to that particular antigen. This process may be used to isolate and concentrate a particular protein or DNA from a sample and requires that the antibody be coupled to a solid substrate at some point in the procedure. The solid substrate includes for example beads, such as magnetic beads. Other types of beads and solid substrates may be used.

For example, a 5-mC antibody (e.g., wherein the 5-mC antibody specifically binds to 5-methylcytosine) may be used as a binder. For the immunoprecipitation procedure, in some embodiments at least 0.05 μg of the antibody is added to the sample, while in some embodiments at least 0.16 μg of the antibody is added to the sample. In some cases, 0.05 μg to 0.80 μg, 0.16 μg to 0.80 μg, 0.40 μg to 0.80 μg, 0.16 μg to 0.40 μg, 0.10 μg to 0.80 μg, 0.20 μg to 0.60 μg, 0.30 μg to 0.50 μg, or 0.40 μg to 0.50 μg of the antibody can be used. To confirm the immunoprecipitation reaction, in some embodiments the method described herein further comprises the operation of adding a second amount of control DNA to the sample.

### Methylation Profile

The present disclosure provides methods, systems, and kits for producing a methylation profile of a subject that has a disease/condition or is suspected of having such disease/condition, wherein the methylation profile may be used to determine whether the subject has the disease/condition or is at risk of having the disease/condition. In some cases, a methylation profile can comprise analysis (e.g., comprising sequencing) of a plurality of nucleic acids (e.g., a plurality of nucleic acid molecules of a depleted sequencing library, as described herein). In some cases, a methylation profile can comprise detection of methylated nucleotides and/or quantification of methylated nucleotide counts, e.g., in a population of nucleic acids of a depleted sequencing library, as described herein. In some cases, a methylation profile can comprise determination of a methylated signal, e.g., in a population of nucleic acids of a depleted sequencing library, as described herein.

### Genomic Mutation Profile

The present disclosure provides methods, systems, and kits for producing a mutation profile of a subject that has a disease/condition or is suspected of having such disease/condition, wherein the methylation profile may be used to determine whether the subject has the disease/condition or is at risk of having the disease/condition. The samples disclosed herein can be subjected to library preparation and next generation deep sequencing, for example to a depth of 1 million (M) to 60 M single reads, 10 M to 60 M single reads, 10 M to 100 M single reads, 40 M to 60 M single reads, 40 M to 100 M single reads, 60 M to 100 M single reads, 60 M to 200 M single reads, 1 M to 10 M single reads, 1 M to 40 M single reads, 1 M single reads to 100 M single reads, 1 M single reads to 200 M single reads, at least 1 M single reads, at least 10 M single reads, at least 40 M single reads, at least 60 M single reads, at least 100 M single reads, or at least 200 M single reads. In some cases, sequencing can be performed at low sequencing depth (e.g., 10 M single reads, 20 M single reads, 30 M single reads, 40 M single reads, from 1 M single reads to 10 M single reads, from 10 M single reads to 20 M single reads, from 20 M single reads to 30 M single reads, from 30 M single reads to 40 M single reads, at most 10 M single reads, at most 20 M single reads, at most 30 M single reads, or at most 40 M single reads). In some cases, a sample disclosed herein can be subjected to 1 sequencing at a depth of 0.1× to 100×, 0.1× to 60×, 0.1× to 40×, 0.1× to 30×, 0.1× to 20×, 0.1× to 10×, 0.1× to 5.0×, 0.5× to 100×, 0.5× to 60×, 0.5× to 40×, 0.5× to 30×, 0.5× to 20×, 0.5× to 10×, 0.5× to 5.0×, 1.0× to 100×, 1.0× to 60×, 1.0× to 40×, 1.0× to 30×, 1.0× to 20×, 1.0× to 10×, 1.0× to 5.0×, at least 0.1×, at least 0.5×, at least 1.0×, at least 2.0×, at least 3.0×, at least 4.0×, at least 5.0×, at least 10.0×, at least 20.0×, at least 30.0×, at least 40.0×, at least 50.0×, at least 60.0×, at least 100×, at least 200×, at most 0.1×, at most 0.5×, at most 1.0×, at most 2.0×, at most 3.0×, at most 4.0×, at most 5.0×, at most 10.0×, at most 20.0×, at most 30.0×, at most 40.0×, at most 50.0×, at most 60.0×, at most 100×, or at most 200×. A plurality of sequencing reads is generated and analyzed. In some embodiments, deep sequencing may be configured to maximize identifying genomic mutations associated with the disease/condition.

In some embodiments, the relative measure of ctDNA abundance is calculated from the mean mutant allele fractions (MAFs). In some embodiments, the mean MAF of mutations identified a subject and comprised in his/her mutation profile ranges from at least about 0.01% to at least about 10%. In some cases, the MAF of a ctDNA fraction of a sample can be about at least 0.01%, 0.02%, 0.03%, 0.04%, 0.05%, 0.06%, 0.07%, 0.08%, 0.09%, 0.1%, 0.15%, 0.2%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or any percentage in between.

In some embodiments, a generated mutation profile of a subject can be generated from sequencing results. In some embodiments, the mutation profile comprises genetic polymorphisms, such as missense variant, a nonsense variant, a deletion variant, an insertion variant, a duplication variant, an inversion variant, a frameshift variant, or a repeat expansion variant. In some embodiments, the mutation profile may comprise mutation variant derived from a fraction of cell-free nucleic acid molecules of a specific size range. The present disclosure provides methods, systems, and kits for producing a mutation profile of a subject that has a disease/condition or is suspected of having such disease/condition, wherein the methylation profile may be used to determine whether the subject has the disease/condition or is at risk of having the disease/condition. Producing a genomic mutation profile can comprise subjecting a plurality of nucleic acid molecules to library preparation and next generation deep sequencing (e.g., MeDIP-seq). A plurality of sequencing reads can be generated and analyzed, and, in some cases, deep sequencing may be configured to maximize identifying genomic mutations associated with the disease/condition. For example, a panel of canonical cancer driver genes may be included in a selector for sequencing results analysis. In some embodiments, including genes without documented driver effects in a particular cancer type in the analysis of sequencing data may increase the sensitivity of ctDNA detection.

In some embodiments, the relative measure of ctDNA abundance is calculated from the mean mutant allele fractions (MAFs). In some embodiments, the mean MAF of mutations identified a subject and comprised in his/her mutation profile ranges from at least about 0.01% to at least about 10%. The ctDNA fraction of a sample disclosed herein is about at least 0.01%, 0.02%, 0.03%, 0.04%, 0.05%, 0.06%, 0.07%, 0.08%, 0.09%, 0.1%, 0.15%, 0.2%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or any percentage in between.

In some embodiments, the generated mutation profile of a subject does not include mutation variants derived from cell-free nucleic acid molecules derived from a biological sample. In some embodiments, the mutation profile comprises genetic polymorphisms, such as missense variant, a nonsense variant, a deletion variant, an insertion variant, a duplication variant, an inversion variant, a frameshift variant, or a repeat expansion variant. In some embodiments, the mutation profile may comprise mutation variant derived from a fraction of cell-free nucleic acid molecules of a specific size range.

### Fragment Length Profile

In some embodiment, the length of ctDNA fragments is shorter than cell-free nucleic acid molecules derived from a healthy subject. In some embodiments, the length of ctDNA comprising at least one mutation is shorter than the length of cell free nucleic acid molecule containing a corresponding reference allele.

In some embodiments, the sequencing does not utilize bisulfite sequence because it causes degradation of ctDNA fragments and prevents the preservation of the length distribution of ctDNAs. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be from 1 to about 800 basepairs (bp), from about 50 bp to about 800 bp, from about 100 bp to about 200 bp, from about 120 bp to about 150 bp, from about 60 to about 500 bp, from about 80 to about 300 bp, from 90 to about 250 bp, from 80 to 170 bp, or from about 100 to about 150 bp. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be at least 800 basepairs (bp), at least 700 basepairs, at least 600 basepairs, at least 500 basepairs, at least 400 basepairs, at least 300 basepairs, at least 200 basepairs, at least 150 basepairs, at least 100 basepairs, or at least 50 basepairs. In some embodiments, the fragment length of a plurality of nucleic acids of the present disclosure (e.g., comprising a mixture cfDNA molecules derived from tumor or cancer tissue and healthy tissue, comprising cfDNA molecules only from healthy tissue, and/or comprising only ctDNA) can be at most 800 basepairs (bp), at most 700 basepairs, at most 600 basepairs, at most 500 basepairs, at most 400 basepairs, at most 300 basepairs, at most 200 basepairs, at most 150 basepairs, at most 100 basepairs, or at most 50 basepairs. In some embodiments, the present disclosure provides an enrichment of the cell free nucleic acid samples based on selecting cell free molecules of a certain size. In some embodiments, the multimodal analysis comprises utilizing the mutation profile described herein and the fragment length profile by selectively including a plurality of nucleic acid molecules in the mutation profile based on their fragment length. In some embodiments, the multimodal analysis comprises utilizing the methylation profile described herein and the fragment length profile by selectively including a plurality of nucleic acid molecules in the methylation profile based on their fragment length. In some embodiments, the multimodal analysis comprises utilizing the mutation profile, methylation profile, and the fragment length profile together by selectively including a plurality of nucleic acid molecules in the mutation profile based on their fragment length and by selectively including a plurality of nucleic acid molecules in the methylation profile based on their fragment length respectively.

### Tumor Detection and Prognosis

The present disclosure provides methods and systems for determining whether a subject has or is at risk of having a disease, wherein the methods and systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the specificity is at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

In some embodiments, the methods and systems can comprise subjecting a plurality of nucleic acid molecules derived from a cell-free nucleic acid sample obtained from said subject to sequencing to generate at least two profiles of (i) a methylation profile, (ii) a mutation profile, and (iii) a fragment length profile. The methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the sensitivity when using two profiles is increased by at least about 0.5%, 1%, 2%, 3%, 4%, 5%, 6%, 7%, 8%, 9%, 10%, or percentage in between any of the numbers compared to the sensitivity when using one profile. In some embodiments, the sensitivity when using three profiles is increased by at least about 0.5%, 1%, 2%, 3%, 4%, 5%, 6%, 7%, 8%, 9%, 10%, or percentage in between any of the numbers compared to the sensitivity when using two profiles.

Further, the methods can provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers. In some embodiments, the specificity when using two profiles is increased by at least about 0.5%, 1%, 2%, 3%, 4%, 5%, 6%, 7%, 8%, 9%, 10%, or percentage in between any of the numbers compared to the specificity when using one profile. In some embodiments, the specificity when using three profiles is increased by at least about 0.5%, 1%, 2%, 3%, 4%, 5%, 6%, 7%, 8%, 9%, 10%, or percentage in between any of the numbers compared to the specificity when using two profiles.

The present disclosure provides methods and systems for processing a cell-free nucleic acid sample of a subject to determine whether said subject has or is at risk of having a disease, the methods and systems comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure provides methods and systems for determining a tissue origin of a tumor, comprising identifying a nucleotide sequence specific for a particular cancer (e.g., breast cancer, colon cancer, prostate cancer, HSNCC, or lung cancer) from which a fraction of cell-free nucleic acid molecules. In some embodiments, the fraction of the cell-free nucleic acid molecules is derived from ctDNA. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure describes methods and systems for providing a prognosis to a subject after receiving a treatment for a disease/condition. For example, the treatment comprises a surgical removal of a tumor, a chemotherapy designed for a specific type of cancer, a radio therapy, or an immune therapy (e.g., TCR, CAR, etc.). In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based at least based on the at least one profile.

Once a subject is accurately diagnosed and receives a treatment to treat the cancer, such as surgical removal, chemotherapy, radio therapy, etc., it can be important to monitor the effectiveness of the treatment and predict the patient's survival rate. Further, it can be important to detect minimal residual disease of cancer cells.

In some embodiments, the method further comprises the operation of adding a second amount of control DNA to the sample for confirming the immunoprecipitation reaction.

As used herein, the “control” may comprise both positive and negative control, or at least a positive control.

In some embodiments, the method further comprises the operation of adding a second amount of control DNA to the sample for confirming the capture of cell-free methylated DNA.

In some embodiments, identifying the presence of DNA from cancer cells further includes identifying the cancer cell tissue of origin.

In some instances, tumor tissue sampling may be challenging or carry significant risks, in which case diagnosing and/or subtyping the cancer without the need for tumor tissue sampling may be desired. For example, lung tumor tissue sampling may require invasive procedures such as mediastinoscopy, thoracotomy, or percutaneous needle biopsy; these procedures may result in a need for hospitalization, chest tube, mechanical ventilation, antibiotics, or other medical interventions. Some individuals may not undergo the invasive procedures needed for tumor tissue sampling either because of medical comorbidities or due to preference. In some instances, the actual procedure for tumor tissue procurement may depend on the suspected cancer subtype. In other instances, cancer subtype may evolve over time within the same individual; serial assessment with invasive tumor tissue sampling procedures is often impractical and not well tolerated by patients. Thus, non-invasive cancer subtyping via blood test may have many advantageous applications in the practice of clinical oncology.

Accordingly, in some embodiments, identifying the cancer cell tissue of origin further includes identifying a cancer subtype. In some cases, the cancer subtype differentiates the cancer based on stage (e.g., early stage lung cancer treated with surgery vs late stage lung cancer treated with chemotherapy), histology (e.g., small cell carcinoma vs adenocarcinoma vs squamous cell carcinoma in lung cancer), gene expression pattern or transcription factor activity (e.g., ER status in breast cancer), copy number aberrations (e.g., HER2 status in breast cancer), specific rearrangements (e.g., FLT3 in AML), specific gene point mutational status (e.g., IDH gene point mutations), and DNA methylation patterns (e.g., MGMT gene promoter methylation in brain cancer).

In some embodiments, comparisons can be carried out genome-wide. In other embodiments, the comparisons can be restricted from genome-wide to specific regulatory regions, such as, but not limited to, long interspersed nuclear elements (LINEs), short interspersed nuclear elements (SINEs), long terminal repeats (LTRs), FANTOM5 enhancers, CpG Islands, CpG shores, CpG Shelves, or any combination of the foregoing.

In some embodiments, the methods herein are for use in the detection of the cancer. In some embodiments, the methods herein are for use in monitoring therapy of the cancer.

### Data Analysis Systems and Methods

The methods and systems disclosed herein may comprise algorithms or uses thereof. The one or more algorithms may be used to classify one or more samples from one or more subjects. The one or more algorithms may be applied to data from one or more samples. The data may comprise biomarker expression data. In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based on at least one profile. The methods disclosed herein may comprise assigning a classification to one or more samples from one or more subjects. Assigning the classification to the sample may comprise applying an algorithm to the methylation profile, mutation profile, and fragment length profile. In some cases, at least one profile is inputted to a data analysis system comprising a trained algorithm for classifying the sample as obtained from a subject which has a disease or minor injuries.

A data analysis system may be a trained algorithm. The algorithm may comprise a linear classifier. In some instances, the linear classifier comprises one or more of linear discriminant analysis, Fisher's linear discriminant, Naïve Bayes classifier, Logistic regression, Perceptron, Support vector machine, or a combination thereof. The linear classifier may be a support vector machine (SVM) algorithm. The algorithm may comprise a two-way classifier. The two-way classifier may comprise one or more decision tree, random forest, Bayesian network, support vector machine, neural network, or logistic regression algorithms.

The algorithm may comprise one or more linear discriminant analysis (LDA), Basic perceptron, Elastic Net, logistic regression, (Kernel) Support Vector Machines (SVM), Diagonal Linear Discriminant Analysis (DLDA), Golub Classifier, Parzen-based, (kernel) Fisher Discriminant Classifier, k-nearest neighbor, Iterative RELIEF, Classification Tree, Maximum Likelihood Classifier, Random Forest, Nearest Centroid, Prediction Analysis of Microarrays (PAM), k-medians clustering, Fuzzy C-Means Clustering, Gaussian mixture models, graded response (GR), Gradient Boosting Method (GBM), Elastic-net logistic regression, logistic regression, or a combination thereof. The algorithm may comprise a Diagonal Linear Discriminant Analysis (DLDA) algorithm. The algorithm may comprise a Nearest Centroid algorithm. The algorithm may comprise a Random Forest algorithm. In some embodiments, for discrimination of preeclampsia and non-preeclampsia, the performance of logistic regression, random forest, and gradient boosting method (GBM) is superior to that of linear discriminant analysis (LDA), neural network, and support vector machine (SVM).

The present disclosure provides methods and systems for determining whether a subject has or is at risk of having a disease, wherein the methods and systems comprises subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and processing said at least one profile to determine whether said subject has or is at risk of said disease at a sensitivity of at least 80% or at a specificity of at least about 90%, wherein said cell-free nucleic acid sample comprises less than 30 ng/ml of said plurality of nucleic acid molecules. In some embodiments, the sensitivity is at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the specificity is at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

In some embodiments, the methods and systems can comprise subjecting a plurality of nucleic acid molecules derived from a cell-free nucleic acid sample obtained from said subject to sequencing to generate at least two profiles of (i) a methylation profile, (ii) a mutation profile, and (iii) a fragment length profile. The methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. In some embodiments, the sensitivity when using two profiles is increased by at least about 0.5%, 1%, 2%, 3%, 4%, 5%, 6%, 7%, 8%, 9%, 10%, or percentage in between any of the numbers compared to the sensitivity when using one profile. In some embodiments, the sensitivity when using three profiles is increased by at least about 0.5%, 1%, 2%, 3%, 4%, 5%, 6%, 7%, 8%, 9%, 10%, or percentage in between any of the numbers compared to the sensitivity when using two profiles.

Further, the methods can provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers. In some embodiments, the specificity when using two profiles is increased by at least about 0.5%, 1%, 2%, 3%, 4%, 5%, 6%, 7%, 8%, 9%, 10%, or percentage in between any of the numbers compared to the specificity when using one profile. In some embodiments, the specificity when using three profiles is increased by at least about 0.5%, 1%, 2%, 3%, 4%, 5%, 6%, 7%, 8%, 9%, 10%, or percentage in between any of the numbers compared to the specificity when using two profiles.

The present disclosure provides methods and systems for processing a cell-free nucleic acid sample of a subject to determine whether said subject has or is at risk of having a disease, the methods and systems comprise providing said cell-free nucleic acid sample comprising a plurality of nucleic acid molecules; subjecting said plurality of nucleic acid molecules or derivatives thereof to sequencing to generate a plurality of sequencing reads; computer processing said plurality of sequencing reads to identify, for said plurality of nucleic acid molecules, (i) a methylation profile, (ii) a mutation profile, and (iii) a fragment length profile; and using at least said methylation profile, said mutation profile and said fragment length profile to determine whether said subject has or is at risk of having said disease. In some embodiments, the methods provide a sensitivity of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or any percentage in between the numbers. The methods can provide a specificity of at least about 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or any percentage in between the numbers.

The present disclosure describes methods and systems for providing a prognosis to a subject after receiving a treatment for a disease/condition. For example, the treatment comprises a surgical removal of a tumor, a chemotherapy designed for a specific type of cancer, a radio therapy, or an immune therapy (e.g., TCR, CAR, etc.). In some embodiments, the methods or systems comprise subjecting a plurality of nucleic acid molecules derived from a cell-free nucleic acid sample obtained from said subject to sequencing to generate at least one profile of (i) a methylation profile, (ii) a mutation profile, and (iii) a fragment length profile; and monitoring or detecting minimal residual disease (MRD) based on the at least one profile.

### Methylation Fraction Fragmentation (MFF) Analysis

As discussed herein, the cancer genome can be globally hypomethylated with focal hypermethylation at CpG Islands as compared to the normal genome. Moreover, circulating tumor DNA (ctDNA) observed in cancer patients can have a shorter fragment length as compared to normal cell-free DNA (cfDNA). Therefore, a method that can capture these shifts in circulating DNA fragment lengths separately at methylated and unmethylated fractions can allow for sensitive cancer detection. Moreover, capturing these shifts in circulating DNA fragment lengths at the unmethylated fraction can allow for sensitive cancer detection at shallow sequencing depth, due to frequently observed global hypomethylation of the cancer genome. A method of using cell-free DNA (cfDNA) fragmentation patterns in methylation fractionated libraries for cancer detection (termed “Methylation Fraction Fragmentation” or “MFF” analysis) can achieve these goals.

In an example, ctDNA is identified by determining occurrence frequencies of short fragments and long fragments in the methylation fractionated libraries. In some cases, regions that are hypomethylated in tumor derived DNA (e.g., ctDNA) can be identified by the presence of an increased frequency of short fragments mapping to that region in the depleted libraries from cancer patients as compared to the depleted libraries of healthy controls. In some cases, regions that are hypermethylated in tumor derived DNA can be identified by the presence of an increased frequency of short fragments mapping to that region in the enriched libraries from cancer patients as compared to the enriched libraries of healthy controls.

Methylation fractionated libraries can comprise sequencing libraries enriched for methylated DNA (e.g., immunoprecipitated methylation “enriched” cfMeDIP-seq libraries). In some cases, methylation fractionated libraries can comprise sequencing libraries depleted for methylated DNA (e.g., “depleted libraries” as described herein, which can comprise cfMeDIP-seq flowthrough). Enriched libraries may be above a threshold methylation level as a result of enrichment of (hyper) methylated DNA or depletion of (hypo) methylated DNA. Depleted libraries may be below a threshold methylation level as a result of enrichment of (hypo) methylated DNA or depletion of (hyper) methylated DNA. MFF analysis can be used to determine the presence or absence of circulating tumor DNA (ctDNA) in a sample of cfDNA obtained from a biological sample, such as one or more biological samples listed herein, such as blood plasma, urine, CSF, etc.

The enriched or depleted sequencing libraries may be subjected to one or more sequencing reactions to generate sequencing data. The sequencing data may comprise one or more sequencing reads of a plurality of nucleic acid molecules or derivatives thereof. The one or more sequencing reactions may comprise one or more of, but are not limited to, sequencing by hybridization (SBH), sequencing by ligation (SBL), chemical sequencing, chain-termination methods (e.g., Sanger sequencing), shotgun sequencing, quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), sequencing by synthesis, ion semiconductor sequencing, nanopore sequencing, single molecule real time (SMRT) sequencing, sequencing by detecting a change in force following hybridization of an oligo. High-throughput sequencing methods, e.g., on cyclic array sequencing using platforms such as Roche 454, Illumina Solexa, AB-SOLID, Helicos, Polonator platforms and the like, can also be utilized. Sequence reads generated by the one or more sequencing reactions may be single end or paired end reads.

The one or more sequencing reactions may be performed at any appropriate depth. In some cases, use of a depleted or enriched library (e.g., a library derived from nucleic acids with a methylation level at or below a threshold methylation level) as described herein may permit sequencing to be performed at a low (shallow) sequencing depth. The sequencing depth may be expressed as a total number of reads, the ratio of the total number of bases obtained by sequencing relative to the size of the genome, or the average number of times each base is measured in the genome. In some cases, the sequencing data are obtained from sequencing performed to a sequencing depth of at least about 0.001×, about 0.01×, about 0.1×, about 0.2×, about 0.3×, about 0.4×, about 0.5×, about 0.6×, about 0.7×, about 0.8×, about 0.9×, about 1×, about 2×, about 3×, about 4×, about 5×, about 6×, about 7×, about 8×, about 9×, about 10×, about 100×, about 1,000×, or more. In some cases, the sequencing data are obtained from sequencing performed to a sequencing depth of no more than about 1,000×, about 100×, about 10×, about 9×, about 8×, about 7×, about 6×, about 5×, about 4×, about 3×, about 2×, about 1×, about 0.9×, about 0.8×, about 0.7×, about 0.6×, about 0.5×, about 0.4×, about 0.3×, about 0.2×, about 0.1×, about 0.01×, about 0.001×, or less. In some cases, the sequencing data are obtained from sequencing performed to a depth between any two of these numbers. In some cases, the sequencing data are obtained from sequencing performed to a sequencing depth of at least about 1 million, 2 million, 3 million, 4 million, 5 million, 6 million, 7 million, 8 million, 9 million, 10 million, about 11 million, about 12 million, about 13 million, about 14 million, about 15 million, about 16 million, about 17 million, about 18 million, about 19 million, about 20 million, about 25 million, about 30 million, about 35 million, about 40 million, about 45 million, about 50 million, about 55 million, about 60 million, about 65 million, about 70 million, about 75 million, about 80 million, about 85 million, about 90 million, about 95 million, about 100 million, about 200 million, about 300 million, 400 million, about 500 million, about 600 million, about 700 million, about 800 million, about 900 million, about 1 billion, or more reads. In some cases, the sequencing data are obtained from sequencing performed to a sequencing depth of no more than about 1 billion, about 900 million, about 800 million, about 700 million, about 600 million, about 500 million, 4 about 00 million, about 300 million, about 200 million, about 100 million, about 95 million, about 90 million, about 85 million, about 80 million, about 75 million, about 70 million, about 65 million, about 60 million, about 55 million, about 50 million, about 45 million, about 40 million, about 35 million, about 30 million, about 25 million, about 20 million, about 19 million, about 18 million, about 17 million, about 16 million, about 15 million, about 14 million, about 13 million, about 12 million, about 11 million, about 10 million, about 9 million, about 8 million, about 7 million, about 6 million, about 5 million, about 4 million, about 3 million, about 2 million, about 1 million, or fewer reads. In some cases, the sequencing data are obtained from sequencing performed to a depth between any two of these numbers.

Sequencing depth may be modulated based on the type of library (e.g., enriched or depleted) and type of reads. For example, sequencing may be relatively shallower (e.g., from about 5 million to about 100 million or more single reads) when performed on a depleted library and relatively deeper (e.g., from about 40 million to about 200 million or more single reads) when performed on an enriched library.

In some cases, sequencing data (e.g., using one or more enriched or depleted libraries as described herein, for example, as analyzed using cfMeDIP-seq) can be used as input for MFF analysis. In some cases, the sequencing library has been enriched for a hypomethylated region. Alternatively, or additionally, the sequencing library has been depleted for a hypermethylated region. The sequencing library may be at or below a threshold methylation level. In some cases, the threshold methylation level can be from 0.1% to 1%, 1% to 5%, 5% to 10%, 10% to 15%, 15% to 20%, 20% to 25%, 25% to 30%, 30% to 35%, 35% to 40%, 40% to 45%, 45% to 50%, 50% to 55%, 55% to 60%, 65% to 70%, 70% to 75%, 75% to 80%, 80% to 85%, 85% to 90%, 95% to 100%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at most 1%, at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, or at most 100%. In some cases, the sequencing data may be derived from a plurality of libraries. In some cases, the sequencing data are derived from 1, 2, 3, 4, 5, 6, 7, 8, 9, 10, 11, 12, 13, 14, 15, 16, 17, 18, 19, 20, 30, or more sequencing libraries. The plurality of sequencing libraries may comprise libraries that are depleted, enriched, or any combination thereof. In an example, the sequencing data comprise data form a sequencing library generated from a depleted library (e.g., that has had one or more nucleic acid molecules comprising a methylated nucleotide removed) and from an enriched library (e.g., generated by cfMeDIP-seq) as described herein.

The sequencing data may be provided in any appropriate format, such as a FASTA or FASTQ file. The sequencing data may be subjected to one or more processing operations to normalize, regularize, or otherwise transform the sequencing data for bioinformatic analysis. In some cases, the raw reads may be trimmed. In some cases, the reads may be aligned to a reference genome, such as a reference human genome (e.g., GRCh38 or GRCh37). In some cases, the aligned reads are stored in one or more BAM files. In some cases, the BAM files are converted to BED files which provide the chromosome, start, and end site for each mapped read. The fragment length of reads within each BED file can extracted and fragments (e.g., that overlap with a background file and any additional regions of interest) can be selected. From these count matrices, the MFF value can be calculated.

Analysis of sequencing data may be restricted to any appropriate subset of a genome. In some cases, the subset comprises the entire genome. In some cases, the subset comprises certain chromosomes or portions thereof. The portion(s) of the genome may correspond to one or genomic features such as specific loci; chromosomes; repeat sections, such as long terminal repeats (LTRs) or short terminal repeats (STRs); long interspersed nuclear elements (LINEs), short nuclear interspersed elements (SINEs), Alu elements; CpG islands; non-CpG island regions, such as CpG island shores; or combinations thereof. In an example, the subset comprises the allosomes of a human genome. In another example, the subset comprises the autosomes of a human genome. In yet another example, the subset comprises CpG islands on the autosomes of a human genome. In still another example, the subset comprises long terminal repeats (LTRs) on the autosomes of a human genome. Still other combinations of features are contemplated herein.

Alternatively, or additionally, analysis of sequence data may be carried out on one or more binned regions of the genome. Binned regions may comprise any appropriate length. In some cases, bins comprise a length of 1 mega base pairs (Mb), 2 Mb, 3 Mb, 4 Mb, 5 Mb, 6 Mb, 7 Mb, 8 Mb, 9 Mb, 10 Mb, or more. In some cases, bins comprise a length of 10 Mb, 9 Mb, 8 Mb, 7 Mb, 6 Mb, 5 Mb, 4 Mb, 3 Mb, 2 Mb, 1 Mb, or less. Binned regions may span the entire genome or any portion thereof (e.g., specific chromosomes or genomic region features as discussed above).

The sequencing data may be subjected to one or more processing operations to generate a fragment length profile as described herein. The one or more processing operations may be carried out by a computer as described herein. In some cases, the fragment length profile comprises a first portion of the sequencing data corresponding to reads of a fragment length below a threshold value. The fragment length profile may additionally comprise a second portion of the sequencing data corresponding to reads of a fragment length above the threshold value. The first and second portions may be combined or transformed into a fragment fraction score.

The threshold value may comprise any appropriate value. The threshold value may be 10 base pairs (bp), 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, 200 bp, 250, bp, 300 bp, 350 bp, 400 bp, 450 bp, 500 bp, 600 bp, 700 bp, 800 bp, 900 bp, 1,000 bp, or more. The threshold value may be between any two of these numbers.

In some cases, the first portion may comprise sequencing reads that fall within a first range or the second portion may comprise sequencing reads that fall within a second range. In some cases, the upper bound of the first range is below the lower bound of the second range. In some cases, the first range and the second range are contiguous. In such cases, the lower bound of the first range may be referred to the first threshold, the upper bound of the first region and the lower bound of the second region may be referred to as the second threshold, and the upper bound of the second region may be referred to as the third threshold. In some cases, the first range and the second range are not contiguous. In some cases, the first range may be from 200 bp to 250 bp, from 150 bp to 200 bp, from 100 bp to 150 bp, from 50 bp to 100 bp, 1 bp to 50 bp, less than 200 bp, or less than 100 bp. The first range may be used for identification of short fragment lengths. In some cases, the second range may be 151 bp to 200 bp, 151 to 220 bp, 150 bp to 200 bp, 200 bp to 250 bp, 250 bp to 300 bp, 300 bp to 350 bp, or 350 bp to 400 bp, larger than 200 bp, larger than 300 bp, or larger than 400 bp. The second range may be used for identification of long fragment lengths. Any appropriate first and second range may be used. In an example, the first range (e.g., short fragment length) is 100 bp-150 bp and the second range (e.g., long fragment length) is 151-200 bp. In another example, the short fragment length is 100 bp-150 bp and the long fragment length is 151-220 bp. In yet another example, the short fragment length is 80 bp-120 bp and the long fragment length is 175 bp to 250 bp. Still other ranges and combinations thereof are possible.

In some cases, the sequencing reads may be partitioned into more than two categories based on fragment length. In some cases, the sequencing reads may be partitioned into one category based on fragment length. The sequencing reads may be portioned into anywhere from 1 to N categories where N is greater than one and less than or equal to the total number of sequencing reads. In some cases, all N categories are contiguous such that there are from N−1 threshold values (if no extreme upper and lower thresholds) to N+1 threshold values (if both an extreme upper and lower threshold are present). In some cases, none of the N categories are contiguous such that there are from 2N−2 (if no extreme upper and lower thresholds) to 2N threshold values (if both an extreme upper and lower threshold are present). In some cases, some of the categories are contiguous with one or more other categories and some of the categories are not contiguous with another category.

The fragment fraction score (e.g., Methylated Fractionated Fragmentation (MFF) score) may be determined based on one or both the first and second portions of the sequencing data. The first or second portions may comprise a copy number based on the total number of reads below or above the threshold value or falling within the corresponding range. The copy number may be converted to a fraction of the total number of reads below or above the threshold or within each of the corresponding ranges. The fraction of reads below the threshold (or falling within the short fragment length range) may be determined by taking a ratio of the copy number of the first portion of sequencing reads (e.g., the portion of sequencing reads below the threshold value or within the short fragment length range) and dividing it by the copy number (e.g., the sum of sequencing reads of the first and second portions). Such a fraction may be termed a short fragment fraction (SFF) herein. The SFF for a given region (e.g., bin) may be written as

$${SFF}_{k} = \frac{{s}_{k}}{{s}_{k} + {l}_{k}}$$

where k is an index corresponding to the given region, sₖ is the number of reads corresponding to the portion below the threshold value or in the short fragment length range, lₖ is the count of reads corresponding to the portion above the threshold value or in the long fragment length range, and SFFₓ is the short fragment fraction for bin k. The fraction of reads above the threshold may be determined by taking a ratio of the copy number of the second portion of sequencing reads (e.g., the portion of sequencing reads above the threshold value or in the long fragment length range) and dividing it by the total copy number (e.g., the sum of sequencing reads of the first and second portions). Such a fraction may be termed a long fragment fraction (LFF) herein. The LFF for a given region (e.g., bin) of the genome may be written as

$${LFF}_{k} = \frac{{l}_{k}}{{s}_{k} + {l}_{k}}$$

where k is an index corresponding to the given region, sₖ is the number of reads corresponding to the portion below the threshold value or in the short fragment length range, l is the count of reads corresponding to the portion above the threshold value or in the long fragment length region, and LFFₓ is the long fragment fraction for bin k.

A fragment fraction score may comprise a Methylated Fractionated Fragmentation (MFF). An MFF score calculation can comprise subtracting the long fragment fraction (LFF) from the short fragment fraction (SFF), viz:

$${MFF}_{k} = {SFF}_{k} - {LFF}_{k}$$

where MFFₓ is the MFF for bin k, SFFₖ is the SFF for bin k, and LFFₓ is the LFF for bin k. In an example, the SFF and LFF are calculated as described above, where the number of fragments between 100-150 bp (sₖ) or 151-220 bp (lₖ) is divided by the number of fragments between 100-220 bp (sₖ+lₖ). As discussed above, in some cases, the calculation can be performed for one or more binned regions (e.g., each defined bin) of the genome or a subsection thereof (e.g., repeat sections such as LTRs, LINEs, or SINEs; CpG islands; or non-CpG island regions such as CpG island shores). Binned regions may comprise any appropriate length. In some cases, bins comprise a length of 1 mega base pairs (Mb), 2 Mb, 3 Mb, 4 Mb, 5 Mb, 6 Mb, 7 Mb, 8 Mb, 9 Mb, 10 Mb, or more. In some cases, bins comprise a length of 10 Mb, 9 Mb, 8 Mb, 7 Mb, 6 Mb, 5 Mb, 4 Mb, 3 Mb, 2 Mb, 1 Mb, or less. Fragment fraction scores for regions comprising a subset of the genome may be combined (e.g., averaged) to characterize the region. For example, a fragment fraction score may be calculated for a given chromosome by averaging all fragment fraction scores from the bins spanning the chromosome or a subset thereof. In another example, a MFF score is calculated for each autosome of a human genome (chromosomes 1 to 22) restricted to CpG shores. In another example, a MFF is calculated for each autosome of a human genomes (chromosome 1 to 22) restricted to LTRs. In another example, a MFF score is calculated for a plurality of 5 Mb bins spanning all chromosomes of a human genome.

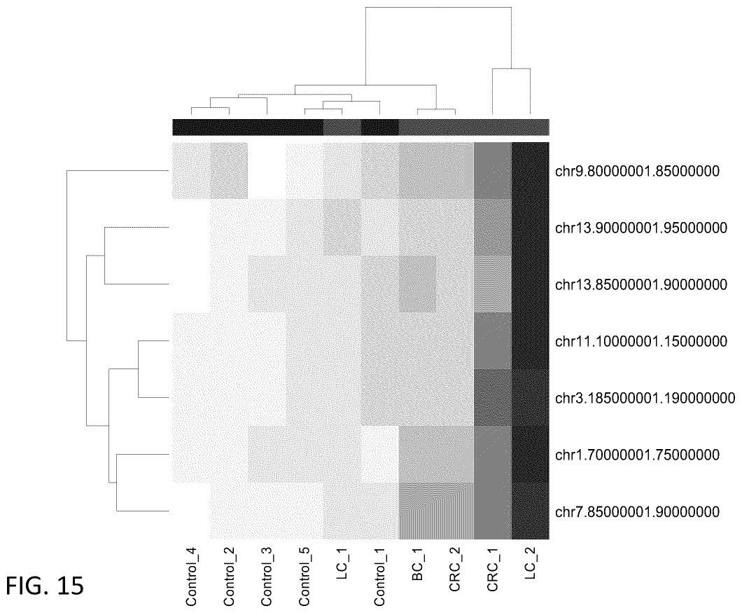

Fragment fraction scores (e.g., MFF scores) may identify genomic regions of interest that have a differential MFF score between cancer and controls in the depleted or enriched libraries (FIG. 15-FIG. 19). Thus, a fragment fraction score may be used to classify a sample (or an individual from which the sample was derived) as belonging to one or more disease-related categories. In some cases, MFF analysis can detect cancer-specific fragmentation patterns at methylated and unmethylated cfDNA fractions. In some cases, MFF analysis can be used to distinguish between populations of nucleic acids (or biological samples from which they are derived) from subjects having cancer and control (e.g., healthy) subjects. In some cases, MFF analysis can be useful even at shallow sequencing (e.g., low sequencing depth). In some cases, improved sensitivity of ctDNA detection by cfMeDIP-seq can be obtained by expanding the repertoire of sequenced ctDNA fragments (i.e., methylated and unmethylated) for detection and subsequent analysis. In some cases, methods as described herein may comprise using a fragment fraction score to determine a likelihood that a nucleic acid sample (or individual from whom the sample was derived) belongs to a disease-related category (e.g., is positive for a disease or condition). For example, a fragment fraction score (e.g., MFF) may be calculated as above. Based on the MFF, a diagnosis of or likelihood of the nucleic acid sample (or individual) being positive for a disease or condition may be made. The determination of likelihood may be made by comparing the MFF at one or more genomic regions to see if they are above or below a certain threshold. In some cases, the determination of likelihood may be made by comparing more than one MFF or a combination or transformation of more than one MFF (e.g., an arithmetic average) at one or more genomic regions. In some cases, the determination is made by one or more algorithms as described herein.

A cutoff or threshold value may be determined by analyzing one or more control samples. Control samples may comprise nucleic acid samples or parts thereof as described herein that are known a priori to be positive for a certain disease or condition (e.g., cancer, such as breast cancer or lung cancer). A cutoff value may be determined by calculating an average fragment fraction score for the control samples. Samples which exhibit a fragment fraction score above (or below) the cutoff value may then be classified accordingly. In some cases, a sample may be classified as having or having an increased likelihood or risk for a disease if an associated fragment fraction score is above the cutoff value. In some cases, a sample may be classified as having or having an increased likelihood or risk for a disease if an associated fragment fraction score is below the cutoff value. In some cases, a sample may be classified as not having or not having an increased likelihood or risk for (e.g., negative for) a disease if an associated fragment fraction score is above the cutoff value. In some cases, a sample may be classified as not having or not having an increased likelihood or risk for (e.g., negative for) a disease if an associated fragment fraction score is below the cutoff value. In an example, a cancer (e.g., breast cancer or lung cancer) is documented to result in hypomethylation of the cancer genome particularly at certain genomic regions (e.g., CpG islands), as compared to normal genomic DNA. Furthermore, circulating tumor DNA (ctDNA) may generally be shorter than other cell-free DNA (cfDNA). A cell-free nucleic acid sample (e.g., blood or fraction thereof, such as plasma; CSF; urine) taken from a subject at risk of or suspected of having a cancer is subjected to operations as described herein to generate a depleted library characterized by methylation below a threshold methylation level. A fragment fraction score (e.g., MFF) is calculated for specific genome regions (e.g., CpG islands on autosomes) and an average MFF is calculated for each chromosome. The MFFs are found, at least on average, to be above the corresponding MFFs from a control sample which is negative for the cancer. Accordingly, the subject is determined to have or be at greater risk for the cancer.

Alternatively, the cutoff value may be determined by calculating a test statistic characterizing the performance of a MFF or combination of MFFs (e.g., an average of MFFs or an MFF at a certain genomic region) at correctly classifying the control data. In some cases, the test statistic may be Youden's Index, F-score, Matthews Correlation Coefficient, phi coefficient, Cohen's kappa, and the like.

Alternatively or additionally, a cutoff may be selected to have a certain accuracy, specificity, sensitivity, or some combination thereof. In an example, the threshold or cutoff value for fragment fraction score (e.g., MFF) may be determined by constructing a receiver operating characteristic curve, and the cutoff is selected as the value which gives the maximal Youden's index for the curve. The control data may comprise nucleic acid samples and known classifications (e.g., positive for a disease, such as cancer) for a set of control samples. Various fragment fraction scores (e.g., at different genomic regions) and combinations thereof (e.g., arithmetic average) may be tested to determine which fragment fraction score or set(s) of fragment fraction scores is the most accurate or otherwise optimal (e.g., as determined by receiver operating characteristic analysis) for determining a likelihood or diagnosis.

In some cases, determining a likelihood (including an increase or decrease thereof) comprises a likelihood of one or more of: a poor clinical outcome, good clinical outcome, high risk of a condition or disease (e.g., a cancer, such as breast or lung cancer), low risk of a condition or disease, complete response, partial response, stable disease, non-response, and recommended treatments for disease management.

In some cases, a fragment fraction score (e.g., MFF) may identify the likelihood of a subject having a disease or belonging to a disease-related category at a high accuracy. In some cases, the accuracy may be about 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher. In some cases, the accuracy is between any two of these numbers. An accuracy may be determined by, for example, comparing a likelihood as determined from a binary classifier to a set of control samples with a known diagnosis or likelihood.

In some cases, a fragment fraction score (e.g., MFF) may identify the likelihood of a subject having a disease or belonging to a disease-related category at a high sensitivity. In some cases, the sensitivity may be about 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher. In some cases, the sensitivity is between any two of these numbers. A sensitivity may be calculated as the percentage of samples positive for a disease-related category (e.g., positive for breast cancer) that are correctly identified as belonging to the disease-related category.

In some cases, a fragment fraction score (e.g., MFF) may identify the likelihood of a subject having a disease or belonging to a disease-related category at a high specificity. In some cases, the specificity may be about 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher. In some cases, the specificity is between any of these numbers. A specificity may be calculated as the percentage of samples negative for a disease-related category (e.g., negative for breast cancer) that are correctly identified as not belonging to the disease-related category.

Methods as disclosed herein may comprise generating one or more reports that are indicative of the one or more fragment length profiles or fragment fraction scores. In some cases, the report may provide a prediction, diagnosis, or prognosis of one or more diseases or health conditions. The one or more reports may comprise a risk of having or developing a disease or condition, status of a disease or condition, prognosis of a disease or health conditions, change in disease or health state, and the like. A therapeutic intervention may be provided upon determining the likelihood of a sample or subject as being positive for a disease or health condition. Non-limiting examples of therapeutic interventions include pharmaceutical compositions, food and diet-based remedies, nutritional supplements, movement based therapies, surgeries, mental and/or cognitive therapies, electro-stimulation therapy, radiation therapy, respiratory therapy, exercise/activity based therapy, phototherapy, and the like. A therapy may be chosen based on the identified disease or health condition in the sample or subject. In some cases, when the disease is a cancer, the treatment may comprise a therapeutically effective dose or amount of surgery, chemotherapy, radiation therapy, targeted therapy, immunotherapy, cell therapy, an antihormonal agent, an antimetabolite chemotherapeutic agent, a kinase inhibitor, a methyltransferase inhibitor, a peptide, a gene therapy, a vaccine, a platinum-based chemotherapeutic agent, an antibody, a checkpoint inhibitor, or any combination thereof.

### Computer Systems

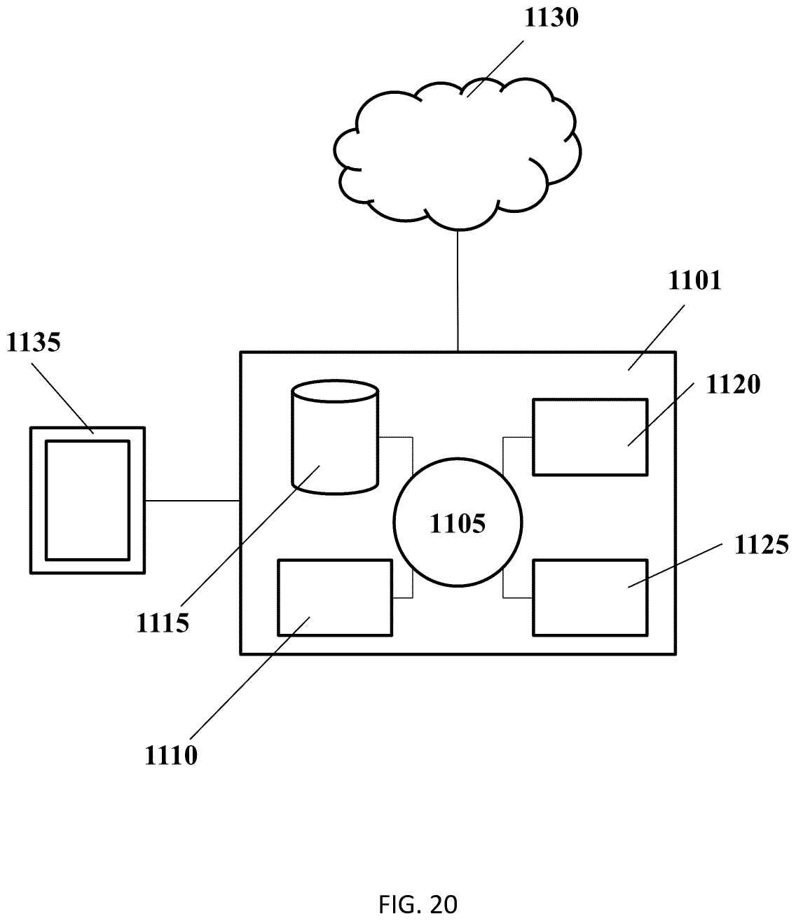

The present disclosure provides computer systems that are programmed to implement methods of the disclosure. FIG. 20 shows a computer system 1101 that is programmed or otherwise configured to generate a sequencing library containing nucleic acid molecules that are depleted of hypermethylated regions of the nucleic acid molecules (e.g., ctDNA). The computer system 1101 can regulate various aspects of the present disclosure. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries, and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### Kits

The present disclosure provides kits for identifying or monitoring a disease or disorder (e.g., cancer) of a subject. A kit may comprise probes for identifying a quantitative measure (e.g., indicative of a presence, absence, or relative amount) of sequences at each of a panel of cancer-associated genomic loci in a sample of the subject. A quantitative measure (e.g., indicative of a presence, absence, or relative amount) of sequences at each of a panel of cancer-associated genomic loci in the sample may be indicative of the disease or disorder (e.g., cancer) of the subject. The probes may be selective for the sequences at the panel of cancer-associated genomic loci in the sample. A kit may comprise instructions for using the probes to process the sample to generate datasets indicative of a quantitative measure (e.g., indicative of a presence, absence, or relative amount) of sequences at each of the panel of cancer-associated genomic loci in a sample of the subject.

The probes in the kit may be selective for the sequences at the panel of cancer-associated genomic loci in the sample. The probes in the kit may be configured to selectively enrich nucleic acid (e.g., RNA or DNA) molecules corresponding to the panel of cancer-associated genomic loci. The probes in the kit may be nucleic acid primers. The probes in the kit may have sequence complementarity with one or more nucleic acid sequences from the panel of cancer-associated genomic loci or genomic regions. The panel of cancer-associated genomic loci or microbiome-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct panel of cancer-associated genomic loci or genomic regions.

The instructions in the kit may comprise instructions to assay the sample using the probes that are selective for the sequences at the panel of cancer-associated genomic loci in the cell-free biological sample. These probes may be nucleic acid molecules (e.g., RNA or DNA) having sequence complementarity with nucleic acid sequences (e.g., RNA or DNA) from one or more of the pluralities of panel of cancer-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of cancer-associated genomic loci in the sample. A quantitative measure (e.g., indicative of a presence, absence, or relative amount) of sequences at each of a panel of cancer-associated genomic loci in the sample may be indicative of a disease or disorder (e.g., cancer).

The instructions in the kit may comprise instructions to measure and interpret assay readouts, which may be quantified at one or more of the panel of cancer-associated genomic loci to generate the datasets indicative of a quantitative measure (e.g., indicative of a presence, absence, or relative amount) of sequences at each of the panel of cancer-associated genomic loci in the sample. For example, quantification of array hybridization or polymerase chain reaction (PCR) corresponding to the panel of cancer-associated genomic loci may generate the datasets indicative of a quantitative measure (e.g., indicative of a presence, absence, or relative amount) of sequences at each of the panel of cancer-associated genomic loci in the sample. Assay readouts may comprise quantitative PCR (qPCR) values, digital PCR (dPCR) values, digital droplet PCR (ddPCR) values, fluorescence values, etc., or normalized values thereof.

### Some Definitions

Various sequencing techniques are known to the person skilled in the art, such as polymerase chain reaction (PCR) followed by Sanger sequencing. Also available are next-generation sequencing (NGS) techniques, also known as high-throughput sequencing, which includes various sequencing technologies including: Illumina (Solexa) sequencing, Roche 454 sequencing, Ion torrent: Proton/PGM sequencing, SOLID sequencing, long reads sequencing (Oxford Nanopore and Pactbio). NGS allow for the sequencing of DNA and RNA much more quickly and cheaply than the previously used Sanger sequencing. In some embodiments, said sequencing is optimized for short read sequencing.

The term “subject” as used herein generally refers to any member of the animal kingdom. Thus, the methods and described herein are applicable to both human and veterinary disease and animal models. Preferred subjects are “patients,” i.e., living humans that are being investigated to determine whether treatment or medical care is needed for a disease or condition; or that are receiving medical care for a disease or condition (e.g., cancer).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The term “nucleic acid” used herein generally refers to a polynucleotide comprising two or more nucleotides, i.e., a polymeric form of nucleotides of any length, either deoxyribonucleotides (dNTPs) or ribonucleotides (rNTPs), or analogs thereof. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, 90%, 95%, or 99%, identity to the nucleotide sequence of the original nucleic acid.

Cell-free methylated DNA is DNA that can be one or more nucleic acid molecules circulating freely in the blood stream. In some cases, cell-free methylated DNA can be methylated at various regions of the DNA. Samples, for example, plasma samples may be taken to analyze cell-free methylated DNA. Studies reveal that much of the circulating nucleic acids in blood arise from necrotic or apoptotic cells and greatly elevated levels of nucleic acids from apoptosis is observed in diseases such as cancer. Particularly for cancer, where the circulating DNA bears hallmark signs of the disease including mutations in oncogenes, microsatellite alterations, and, for certain cancers, viral genomic sequences, DNA or RNA in plasma has become increasingly studied as a potential biomarker for disease. For example, a quantitative assay for low levels of circulating tumor DNA in total circulating DNA may serve as a better marker for detecting the relapse of colorectal cancer compared with carcinoembryonic antigen, the standard biomarker used clinically. Cell-free DNA (e.g., circulating cfDNA) may comprise circulating tumor DNA (ctDNA).

As used herein, “library preparation” generally includes one or more of list end-repair, A-tailing, adapter ligation, or any other preparation performed on the cell free DNA to permit subsequent sequencing of DNA.

As used herein, “supplemental processed DNA” (e.g., “filler DNA”) may be noncoding DNA or it may consist of amplicons.

In some embodiments, the fragment length metric is fragment length. In some preferable embodiments, the subject cell-free methylated DNA is limited to fragments having a length of <170 bp, <165 bp, <160 bp, <155 bp, <150 bp, <145 bp, <140 bp, <135 bp, <130 bp, <125 bp, <120 bp, <115 bp, <110 bp, <105 bp, or <100 bp. In other preferable embodiments, the subject cell-free methylated DNA is limited to fragments having a length of between about 100-about 150 bp, 110-140 bp, or 120-130 bp.

In some embodiments, the fragment length metric is the fragment length distribution of the subject cell-free methylated DNA. In some preferable embodiments, the subject cell-free methylated DNA is limited to fragments within the bottom 50th, 45th, 40th, 35th, 30th, 25th, 20th, 15th, or 10th percentile based on length.

### EXAMPLES

### Example 1: Provision of Cell-Free DNA

This example shows examples of methods and systems for the provision of cell-free DNA, which can be used with or in methods, compositions, systems, and kits used in DNA library creation and/or in determination of a risk in a subject of having a tumor.

Whole blood samples were collected from healthy subjects and subjects diagnosed with a tumor or cancer. For example, methods and systems described herein have been tested using samples obtained from subjects having breast cancer, colorectal cancer, or lung cancer. In some cases, patients had been identified as having an early-stage cancer. In some cases, subjects had been identified as having a late-stage cancer. In some cases (e.g., in breast cancer), early-stage cancer can include in situ, stage I, stage II (for instance stage IIA or stage IIB), or stage IIIA cancer. In some cases, (e.g., in breast cancer), late-stage cancer can include stage IIIB or stage IV cancer.

Plasma was isolated from whole blood within 1 hour of collection and stored at −80° C. until further processing. If freshly drawn whole blood from healthy subjects is unavailable, commercially available normal donor plasma (Cedarlane) or cancer subject plasma can be used. Cell-free DNA (cfDNA) was isolated from 1 to 3 mL total plasma using the Apostle MiniMax High Efficiency cfDNA Isolation Kit (Apostle) or QIAamp Circulating Nucleic Acid Kit (Qiagen) following manufacturer's instructions. In some cases, “cfDNA mimic” was created by shearing commercially obtained K562 genomic DNA (Promega) or HCT116 to lengths of from 150 to 200 base-pairs (bp) using a Covaris LE220 Focused-ultrasonicator, and size-selected by AMPure XP magnetic beads (Beckman Coulter), using a bead ratio of 1.2× to 1.7× (e.g., to remove fragments above 300 base-pairs and under 100 base-pairs). Isolated cfDNA and sheared PBL genomic DNA. cfDNA isolated from subject plasma samples (native cfDNA) and cfDNA mimic were quantified by Qubit prior to library generation. Isolated cfDNA was also profiled using Agilent TapeStation cfDNA Assay Kit to ensure the percent cfDNA (% cfDNA) in isolated cfDNA aliquots was at least 50% (≥50%).

### Example 2: In Vitro DNA Methylation of Supplemental Processed DNA

This example shows examples of methods and systems for in vitro methylation of supplemental processed DNA, for example, to provide nucleic acid material for cfMeDIP immunoprecipitation, library creation, and/or sequencing.

Supplemental processed DNA was prepared as follows: Enterobacteria phage 2 DNA (ThermoFisher Scientific) was amplified using the primers indicated in Table 1, generating 6 different PCR amplicons products. The PCR reaction was carried out using Platinum Superfi PCR mastermix with the following condition: activation of enzyme at 98° C. for 30 seconds (sec), 30 cycles of: 98° C. for 1 sec, 57° C. for 10 sec, 72° C. for 15 sec and a final extension at 72° C. for 5 min. The PCR amplicons were purified with QIAQuick PCR purification kit (Qiagen) and ran on a gel to verify size and amplification. Amplicons for 1CpG, 5CpG, 10CpG, 15CpG and 20CpGL were methylated using CpG Methyltransferase (M.SssI) (ThermoFisher Scientific) and purified with the QIAQuick PCR purification kit. Methylation of the PCR amplicons was tested using restriction enzyme HpyCH4IV (New England Biolabs Canada) and ran on a gel to ensure its methylation. The DNA concentration of the unmethylated (20CpGS) and methylated (1CpG, 5CpG, 10CpG, 15CpG, 20CpGL) amplicons was measured using picogreen or Qubit prior to pooling with 50% of methylated and 50% unmethylated λ PCR product.

Methylation reaction using 150 ng of supplemental processed DNA as the starting material was set up using CpG Methyltransferase (M.SssI) (ThermoFisher Scientific, Cat #EM0821), following the manufacturer's protocol. A surrogate control sample was also set up alongside the supplemental processed DNA to test for proper methylation. This surrogate control sample, an amplicon generated in-house which was available in larger quantities, has a restriction site that corresponds to methylation-sensitive restriction enzyme HpyCH4IV. For the in vitro methylation, the volume of the starting material was supplemented to 16.6 μL with nuclease-free water before it was mixed with the following mastermix: 2 μL of 10× M.SssI Buffer, 0.4 μL 50×SAM and 1 μL of M.SssI Enzyme. The reaction was incubated at 37° C. for 15 min, followed by inactivation at 65° C. for 20 min. The methylated DNA was purified using Qiagen MinElute PCR Clean up kit (Qiagen, Cat #28004) following manufacturer's protocol before being quantified via Qubit.

The methylated surrogate control sample and an aliquot of the original surrogate control sample were subjected to methylation sensitive restriction digest using restriction enzyme HpyCH4IV (NEB, Cat #R0619S) following manufacturer's protocol. After purification of the digested product using the Qiagen MinElute PCR Clean up kit, through TapeStation profile, it was verified that there was digestion of the original surrogate sample (multiple smaller products) but no digestion of the methylated surrogate control (single larger product) indicating successful in vitro methylation.

### Example 3: Preparation of Depleted Sequencing Libraries

This example shows examples of methods and systems for the creation of depleted sequencing nucleic acid libraries for the detection of ctDNA in a cfDNA sample and determination of risk of cancer in a subject.

Ten nanograms of input cfDNA (e.g., native cfDNA or DNA mimic) was prepared for library generation using the KAPA HyperPrep Kit (KAPA Biosystems) with some modifications. In some cases, between 1 ng and 10 ng of input cfDNA can be used. For cfDNA extracted from samples obtained from healthy subjects and those diagnosed with cancer (e.g., native cfDNA), 0.1 ng of spike-in control DNA (fully methylated or fully unmethylated synthetic control nucleic acid fragments; Adela) was added. Library sequencing adapters (IDT xGen CS Adapter) comprising unique molecular identifiers according to manufacturer's instructions, with modifications were added to the DNA. Briefly, after end-repair and A-tailing, 0.327 μM xGen CS adapter was ligated to the DNA following an incubation of 30 minutes at 20° C. After post-ligation cleanup, input DNA was eluted in 40 μL of elution buffer (EB, 10 mM Tris-HCl, pH 8.0-8.5) prior to library generation. Additional library preparation steps and conditions, which may be used in place of or in addition to those presented here, can be found in Shen et al. Nat. Protoc. 2019 October; 14 (10): 2749-2780, which is incorporated in its entirety by reference for all purposes, including methods, systems, and compositions used in MeDIP immunoprecipitation.

In some cases, adapter-ligated DNA was combined with supplemental processed DNA to increase starting input DNA into the immunoprecipitation reaction to 100 ng. In some cases, experiments are performed without addition of lambda (λ) supplemental processed DNA. When supplemental processed DNA is used, the supplemental processed DNA is selected from unmethylated DNA (0% methylation), fully methylated DNA (100% methylation), intermediately methylated DNA, or a combination thereof. For example, a mixture of unmethylated supplemental processed DNA and fully methylated DNA is prepared for combination with the input adapter-ligated cfDNA (e.g., to bring immunoprecipitation reaction DNA mass to 100 ng). The ratio of unmethylated supplemental processed DNA to fully methylated DNA can be adjusted to a desired value. For instance, a lower percentage of methylated DNA in the supplemental processed DNA (e.g., a higher percentage of unmethylated DNA) was observed to produce a stronger depletion of methylated cfDNA (e.g., with a constant concentration of 5-methylcytosine binder, such as a 5mC antibody, since the lower percentage of methylated DNA increases the availability of binder to pull down methylated cfDNA fragments from the sample).

The resulting sample comprising adapter-ligated cfDNA (e.g., for experiments with or without utilization of supplemental processed DNA) is combined with immunoprecipitation buffers prior to being heat-denatured and snap-chilled (e.g., to convert DNA into single-stranded configurations, which improves capture by the binder). This heat-denaturation operation may be used with certain 5-methylcytosine-specific immunoprecipitation binders (e.g., some 5-methylcytosine (5mC) antibodies) that are selective for single-stranded DNA for effective pull-down. In some experimental protocols (e.g., wherein the 5mC-specific binder (e.g., a methylated binding protein) can bind to double-stranded DNA and does not require single-stranded DNA for effective pull-down), the heat-denaturation operation can be omitted. In these experiments, a 5mC antibody selective for single-stranded DNA was used, and antibody working concentration was empirically determined. In cases where stronger depletion of methylated cfDNA was desired or required (e.g., wherein sequencing results showed poor or moderate separation of unmethylated cfDNA), the concentration of the 5-methylcytosine-specific binder was increased.

The adapter-ligated cfDNA sample (with or without supplemental processed DNA) and immunoprecipitation buffer mix was incubated with the 5mC-specific binder, and the flow-through was collected. The collected flow-through DNA was purified using a Zymo RNA Clean & Concentrator™-5 kit. Briefly, the flow-through DNA was diluted 1:1 with water and then purified according to the manufacturer's instructions. AMPure XP beads can also be used for purification. This purified DNA was depleted of methylated DNA species and was subsequently indexed and amplified to generate a “depleted library.” The adapter-ligated cfDNA sample retained by the 5mC-specific binder was eluted separately and purified. This purified DNA was enriched for methylated DNA species and was subsequently indexed and amplified to generate an “enriched library.” Five percent (5%) of each group of DNA was saved as an input control.

Amplification was performed with polymerase chain reaction (PCR) mastermix reagents and PCR cycles set to 15 cycles using IDT xGen UDI primers. In the case of input control DNA, amplification was performed using PCR mastermix reagents; however, PCR cycle number was set to 10 cycles. After amplification, both the depleted library and the enriched library were subjected to dual size selection using AMPure XP beads at a 0.6× to 1.0× ratio to remove any remaining primer molecules. For libraries obtained from native cfDNA samples, amplification was performed for 14 cycles before purification with AMPure XP beads. Library samples were then quantified using Qubit (or an alternative size selection protocol) and profiled via TapeStation to verify proper fragment size distribution and DNA quantity.

### Example 4: Sequencing of Cell-Free DNA (cfDNA) from Healthy Subjects and Subjects Having Cancer

This example shows examples of methods and systems for sequencing methylation depleted and methylation enriched nucleic acid libraries.

Depleted and enriched libraries created from blood plasma samples obtained from healthy subjects and subjects having cancer as described in preceding Libraries were normalized and sequenced on an Illumina NovaSeq 6000 sequencer with a paired-end 100 bp (2×100) configuration. It is noted that other sequencers utilizing pair-end capture (e.g., Illumina NextSeq and Illumina HiSeq4000 systems) may be used. Depleted libraries were sequenced at a depth of 10 million single reads (e.g., low sequencing depth), and enriched libraries were sequenced at a depth of 60 million single reads. It is noted that a relatively shallow sequencing depth was used for these experiments, but the depth of sequencing can be selected from a range of 5 million single reads to 100 million single reads (or more than 100 million single reads) for depleted libraries and 40 million single reads to 200 million single reads (or more than 200 million single reads), depending on the specific application.

### Example 5: Sequencing Data Analysis

This example shows examples of methods and systems for in vitro methylation of native cfDNA and cfDNA mimic, for example, to provide nucleic acid material for cfMeDIP immunoprecipitation, library creation, and/or sequencing.

Sequencing results from experiments performed according to protocols outlined in Example 4 and using 5mC antibodies from two different vendors were processed in a bioinformatics pipeline configured to align sample reads with fully methylated or fully unmethylated synthetic control nucleic acid fragments (“spike-ins”, Adela) and with human genome build hg38. Deduplication of reads was performed to remove PCR duplicates from the alignment results. The spike-ins' pull-downs were evaluated by normalizing deduplicated count results by the sum of the spike-in read counts after deduplication and the hg38 read counts after deduplication. Methylation specificities were calculated by dividing fully methylated spike-in counts following deduplication by the sum of the fully methylated spike-in counts and the fully unmethylated spike-in counts.

The first five base-pairs on each 5′ end of unaligned paired reads, corresponding to the incorporated 3 base-pair or 4 base-pair random molecular barcodes, were extracted and collated to generate a 10-bp molecular identifier (UMI). In cases where the incorporated UMIs were three base-pairs on either 5′ end of unaligned paired reads, the fourth T base-pair spacer and fifth base-pair corresponding to the first base-pair of the cfDNA sequence was also incorporated prior to alignment. In cases where the incorporated UMIs were four base-pairs on either 5′ end of unaligned paired reads, the fifth T base-pair spacer was also incorporated. Paired reads were aligned to spike-in sequences by Bowtie2, then sorted and indexed using SAMtools. Duplicate paired reads from aligned spike-ins were removed based on UMIs prior to quantification. Reads with no alignment to spike-in sequences were aligned to the human genome (build hg38) by Bowtie2 and then sorted and indexed using SAMtools. Duplicate paired reads aligned to the human genome were removed based on genome position and UMIs. Quality control of each library was assessed by various metrics obtained from the R package MEDIPS including CpG coverage (MEDIPS.seqCoverage) and enrichment (MEDIPS.CpGenrich).

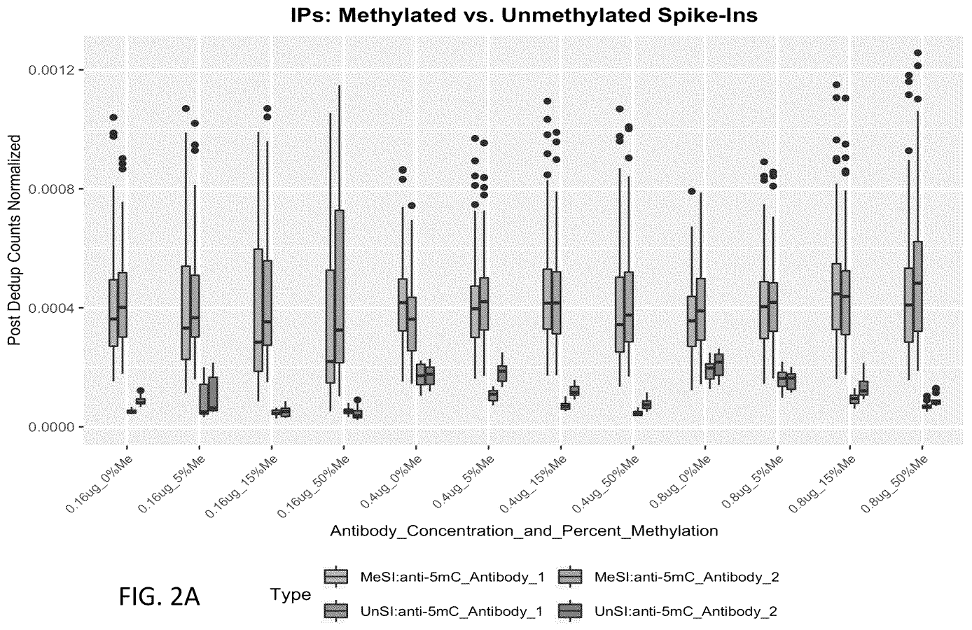

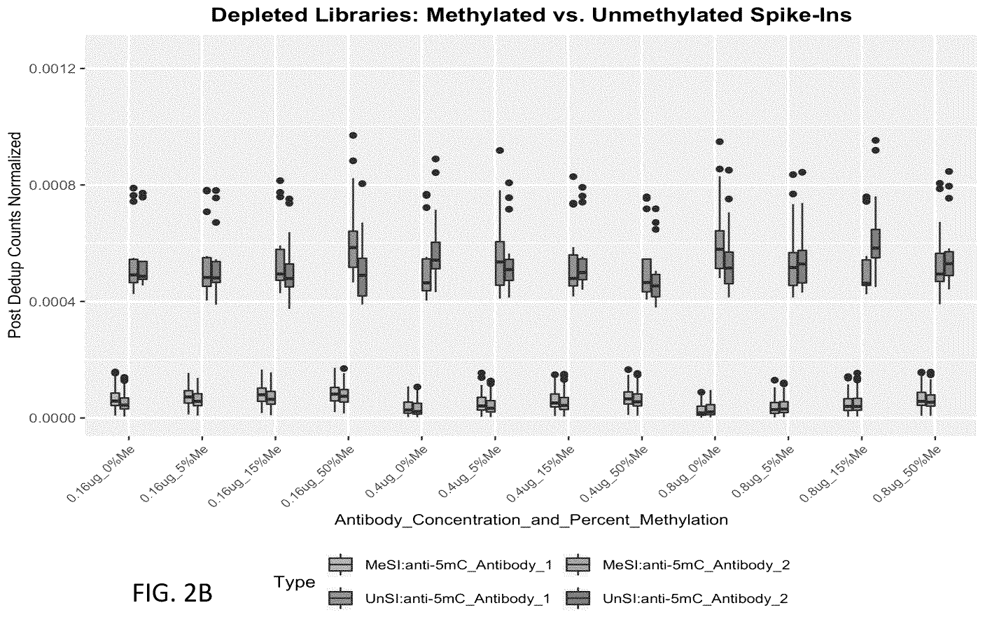

FIG. 2A shows normalized counts for 5mC-enriched libraries (“IPs”) after deduplication (y-axis) across 12 antibody concentrations of each of the two tested antibodies and supplemental processed DNA percentage conditions (x-axis, from left to right: 0.16 micrograms (μg)/0% methylated supplemental processed DNA; 0.16 μg/5% methylated supplemental processed DNA; 0.16 μg antibody/15% methylated supplemental processed DNA; 0.16 μg antibody/50% methylated supplemental processed DNA; 0.4 μg antibody/0% methylated supplemental processed DNA; 0.4 μg antibody/5% methylated supplemental processed DNA; 0.4 μg antibody/15% methylated supplemental processed DNA; 0.4 μg antibody/50% methylated supplemental processed DNA; 0.8 μg antibody/0% methylated supplemental processed DNA; 0.8 μg antibody/5% methylated supplemental processed DNA; 0.8 μg antibody/15% methylated supplemental processed DNA; 0.8 μg antibody/50% methylated supplemental processed DNA). For each condition along the x-axis, bars (from left to right in each condition group) represent data obtained with methylated spike-in using Antibody 1, data obtained with methylated spike-in using Antibody 2, data obtained with unmethylated spike-in using Antibody 1, and data obtained with unmethylated spike-in using Antibody 2 (“MeSI” represents “methylated spike-in samples” while “UnSI” represents “unmethylated spike-in samples”).

FIG. 2B shows normalized counts for 5mC-depleted libraries (“Depleted Libraries”) after deduplication (y-axis) across 12 antibody concentrations of each of the two tested antibodies and supplemental processed DNA percentage conditions (x-axis, from left to right: 0.16 micrograms (μg)/0% methylated supplemental processed DNA; 0.16 μg/5% methylated supplemental processed DNA; 0.16 μg antibody/15% methylated supplemental processed DNA; 0.16 μg antibody/50% methylated supplemental processed DNA; 0.4 μg antibody/0% methylated supplemental processed DNA; 0.4 μg antibody/5% methylated supplemental processed DNA; 0.4 μg antibody/15% methylated supplemental processed DNA; 0.4 μg antibody/50% methylated supplemental processed DNA; 0.8 μg antibody/0% methylated supplemental processed DNA; 0.8 μg antibody/5% methylated supplemental processed DNA; 0.8 μg antibody/15% methylated supplemental processed DNA; 0.8 μg antibody/50% methylated supplemental processed DNA). Once again, for each condition along the x-axis, bars (from left to right in each condition group) represent data obtained with methylated spike-in using Antibody 1, data obtained with methylated spike-in using Antibody 2, data obtained with unmethylated spike-in using Antibody 1, and data obtained with unmethylated spike-in using Antibody 2 (“MeSI” represents “methylated spike-in samples” while “UnSI” represents “unmethylated spike-in samples”).

In each condition, enriched libraries showed higher counts for methylated spike-in experiments than unmethylated spike-in experiments (FIG. 2A). In contrast, depleted libraries showed higher counts for unmethylated spike-in experiments than methylated spike-in experiments (FIG. 2B). Accordingly, depleted libraries were verified as being comprised of mainly unmethylated DNA using the methods and systems disclosed herein.

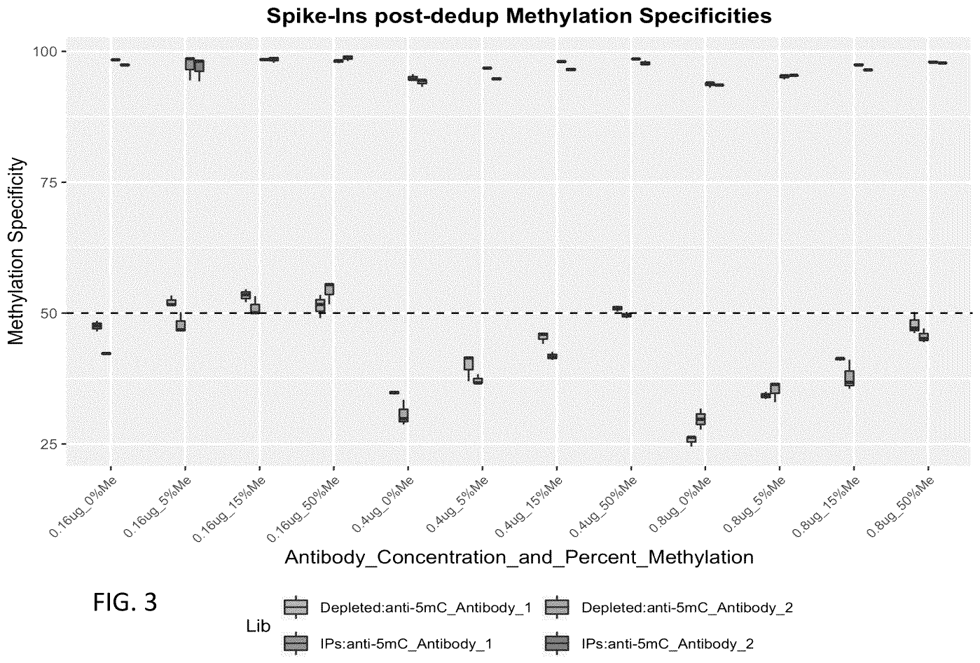

Methylation specificities were found to be far higher for enriched libraries (ranging from 93.06% to 99.24%; mean 96.77%) than for depleted libraries (24.49% to 55.67%; mean 42.82%) across all tested conditions (FIG. 3), showing that the enriched libraries were indeed strongly enriched for methylated nucleic acid fragments while the depleted libraries were strongly depleted for methylated nucleic acid fragments.

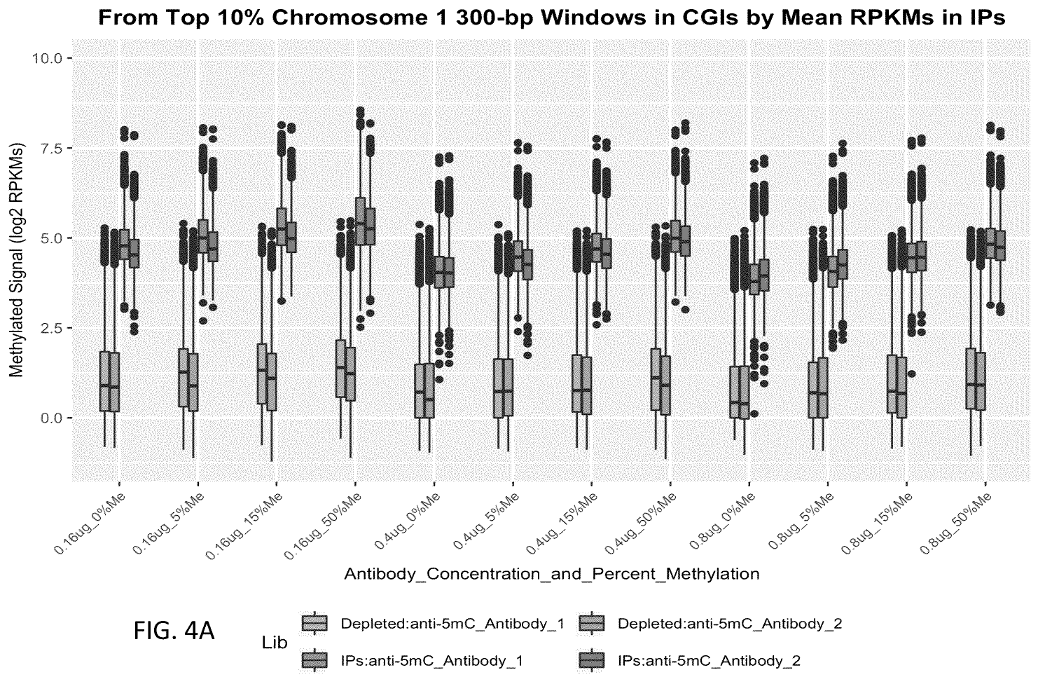

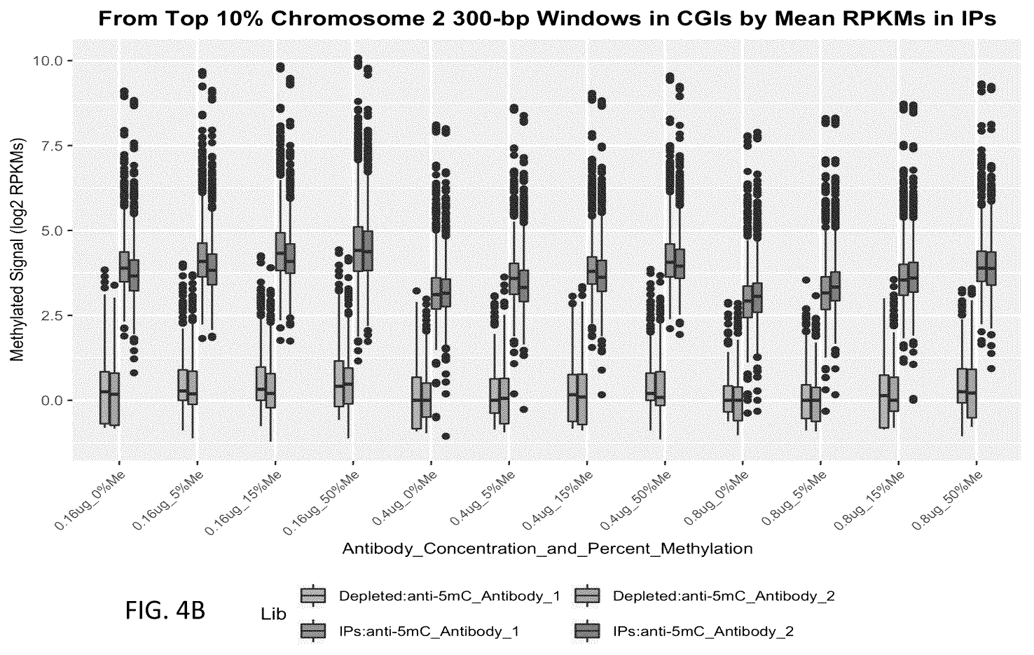

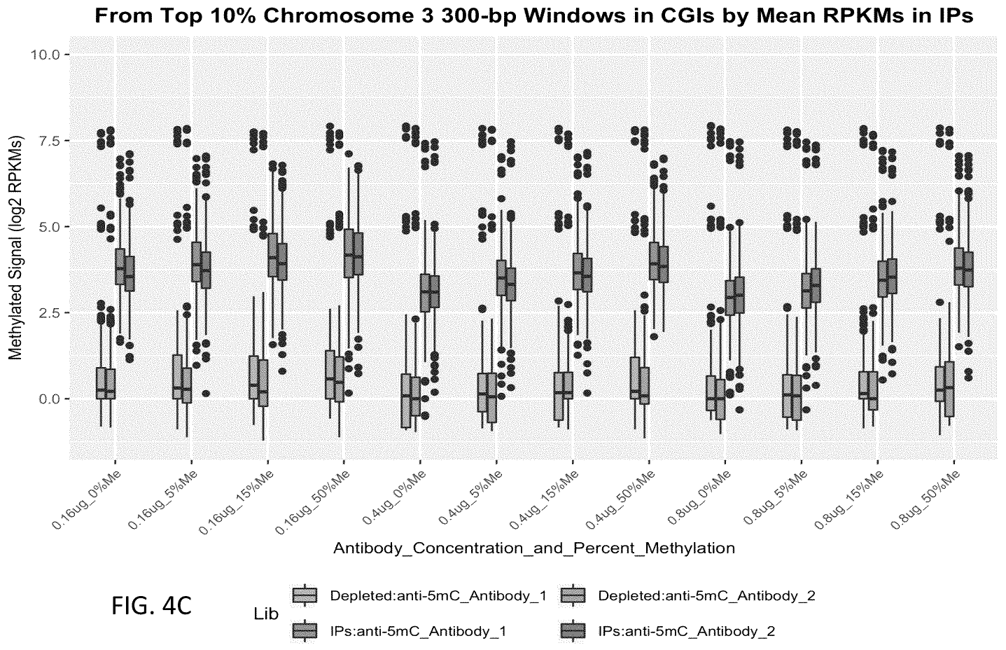

When enriched and depleted libraries created from human cfDNA were compared to human genome build hg38 at three individual chromosomes (as shown in FIGS. 4A, 4B, and 4C), a stronger signal (y-axis, log 2 reads per kilobase per million (RPKM)) was observed for enriched libraries and lower signal was observed for depleted libraries for both anti-5mC antibodies and all antibody concentration and supplemental processed DNA percentages tested (x-axis, from left to right: 0.16 micrograms (μg)/0% methylated supplemental processed DNA; 0.16 μg/5% methylated supplemental processed DNA; 0.16 μg antibody/15% methylated supplemental processed DNA; 0.16 μg antibody/50% methylated supplemental processed DNA; 0.4 μg antibody/0% methylated supplemental processed DNA; 0.4 μg antibody/5% methylated supplemental processed DNA; 0.4 μg antibody/15% methylated supplemental processed DNA; 0.4 μg antibody/50% methylated supplemental processed DNA; 0.8 μg antibody/0% methylated supplemental processed DNA; 0.8 μg antibody/5% methylated supplemental processed DNA; 0.8 μg antibody/15% methylated supplemental processed DNA; 0.8 μg antibody/50% methylated supplemental processed DNA). To quantify the relative methylated signal from cfDNA, non-overlapping windows 300-bp in length were selected across chromosomes 1 to 22 to encompass the range of fragment lengths observed in cfDNA. Fragments generated from paired reads of cfMeDIP-seq libraries were counted within non-overlapping 300 base-pair windows by MEDIPS (MEDIPS.createSet), and the RPKMs (Reads Per Kilobase per Million reads), for each sample were extracted by the MEDIPS.meth function and collated as a matrix into an Rds object.

All 8,971 300-basepair (bp) windows that overlapped CpG Islands (CGIs) on chromosome 1 were examined for each antibody and test condition, and the top 10% (898 windows in total) of RPKM were identified based on mean RPKMs. FIG. 4A shows that enriched libraries (“IPs”, shown as the third and fourth of four box plots for each condition) had a substantially higher methylated signal than depleted libraries (“Depleted”, shown as the first and second box plots for each condition) across all conditions. Similar results were obtained when the top 10% of 300-bp windows were evaluated for chromosome 2 (FIG. 4B), wherein substantially higher methylated signal was observed for enriched libraries than for depleted libraries, across all tested conditions. Results from the top 10% of 300-bp windows of chromosome 3 (FIG. 4C) also showed that substantially higher methylated signal was observed for enriched libraries than for depleted libraries, across all tested conditions.

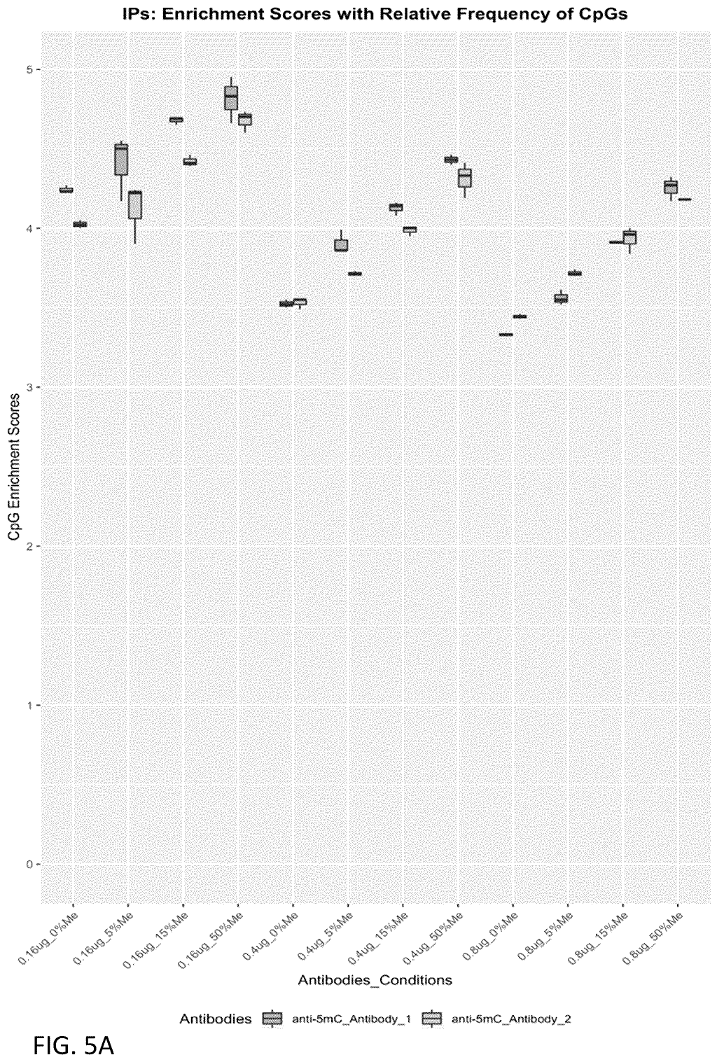

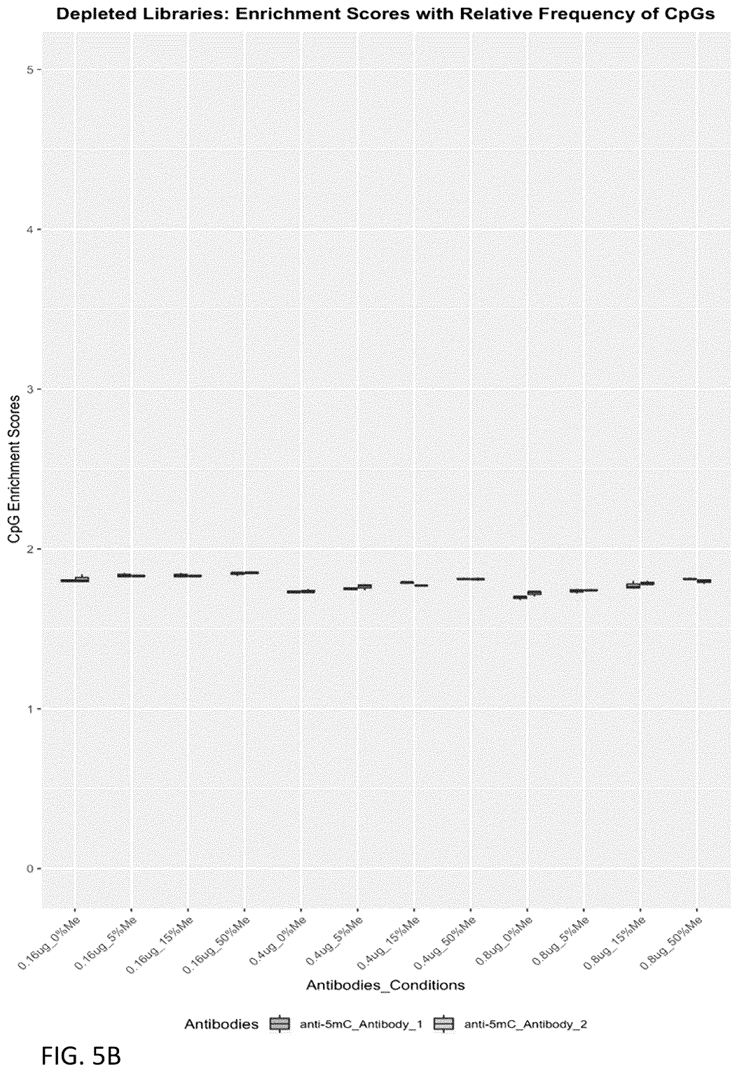

The relative number of CpGs across aligned fragments and the reference genome were calculated by the number of CpG di-nucleotide motifs, divided by the total number of nucleotides across all aligned fragments and the reference genome respectively, multiplied by 100. The CpG enrichment score was subsequently calculated from the relative number of CpGs across aligned fragments, divided by the relative number of CpGs across the reference genome. CpG enrichment scores were calculated for enriched libraries (FIG. 5A) and depleted libraries (FIG. 5B) for both antibodies tested (left box plot for each condition: anti-5mC Antibody 1; right box plot for each condition: anti-5mC Antibody 2) and all antibody concentration and supplemental processed DNA methylation percentage conditions (x-axis, from left to right: 0.16 micrograms (μg)/0% methylated supplemental processed DNA; 0.16 μg/5% methylated supplemental processed DNA; 0.16 μg antibody/15% methylated supplemental processed DNA; 0.16 μg antibody/50% methylated supplemental processed DNA; 0.4 μg antibody/0% methylated supplemental processed DNA; 0.4 μg antibody/5% methylated supplemental processed DNA; 0.4 μg antibody/15% methylated supplemental processed DNA; 0.4 μg antibody/50% methylated supplemental processed DNA; 0.8 μg antibody/0% methylated supplemental processed DNA; 0.8 μg antibody/5% methylated supplemental processed DNA; 0.8 μg antibody/15% methylated supplemental processed DNA; 0.8 μg antibody/50% methylated supplemental processed DNA). Briefly, CpG enrichment score was calculated by dividing the relative frequency of CpGs of the analyzed regions by the relative frequency of CpGs of the human genome. Depleted libraries showed a lower enrichment score for each antibody and each antibody concentration/supplemental processed DNA methylation percentage condition tested. In these experiments, CpG enrichment scores for all tested conditions were less than 2. CpG enrichment scores for enriched libraries were all above 3. Thus, depleted libraries with CpG enrichment scores of 3, below 3, 2, below 2, 1, or below 1 could all be distinguished from enriched libraries. In some cases, for example when 50% methylated supplemental processed DNA was used, it would be possible to distinguish a depleted library having an enrichment score of 4 or below 4 from enriched libraries.

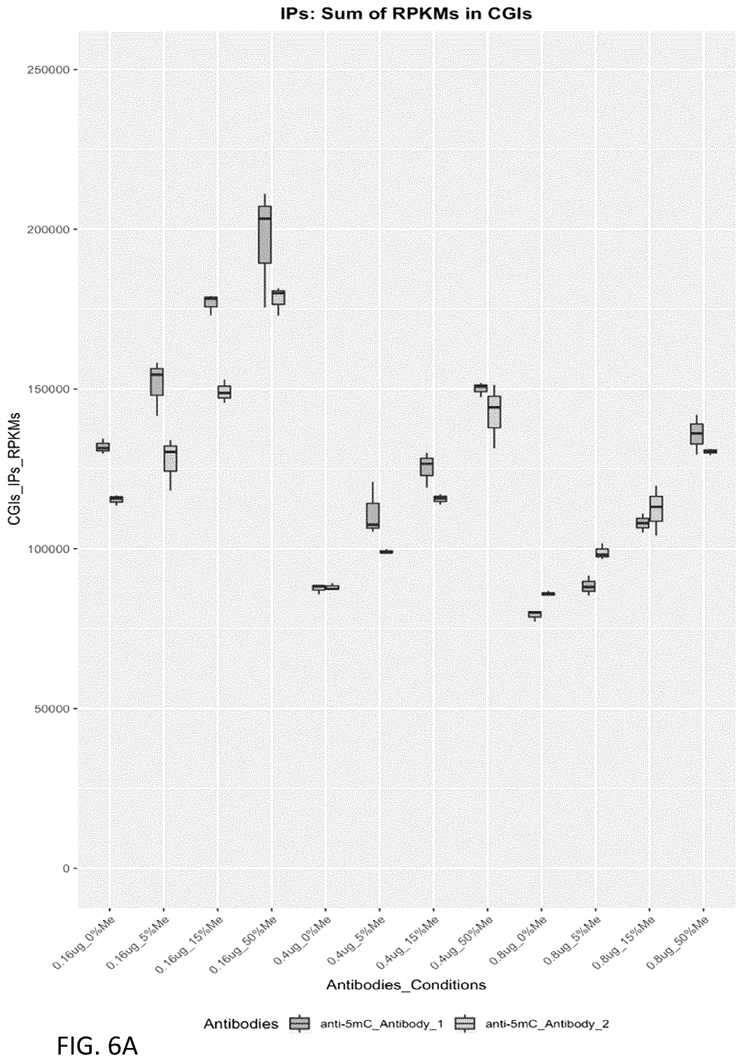

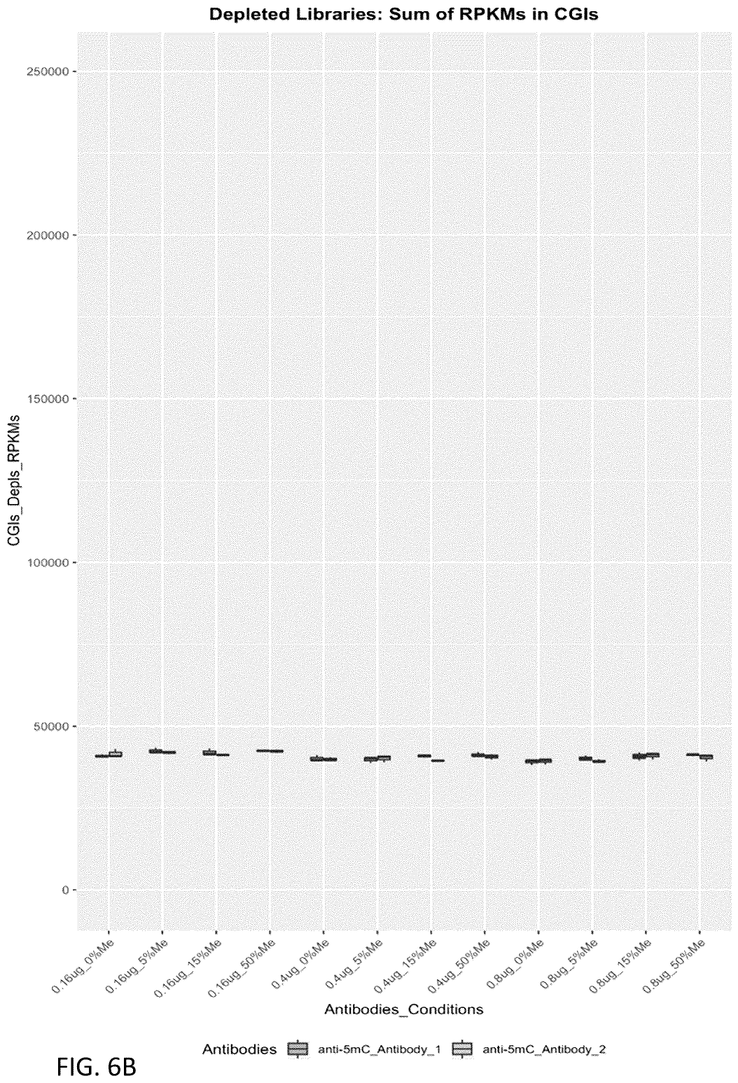

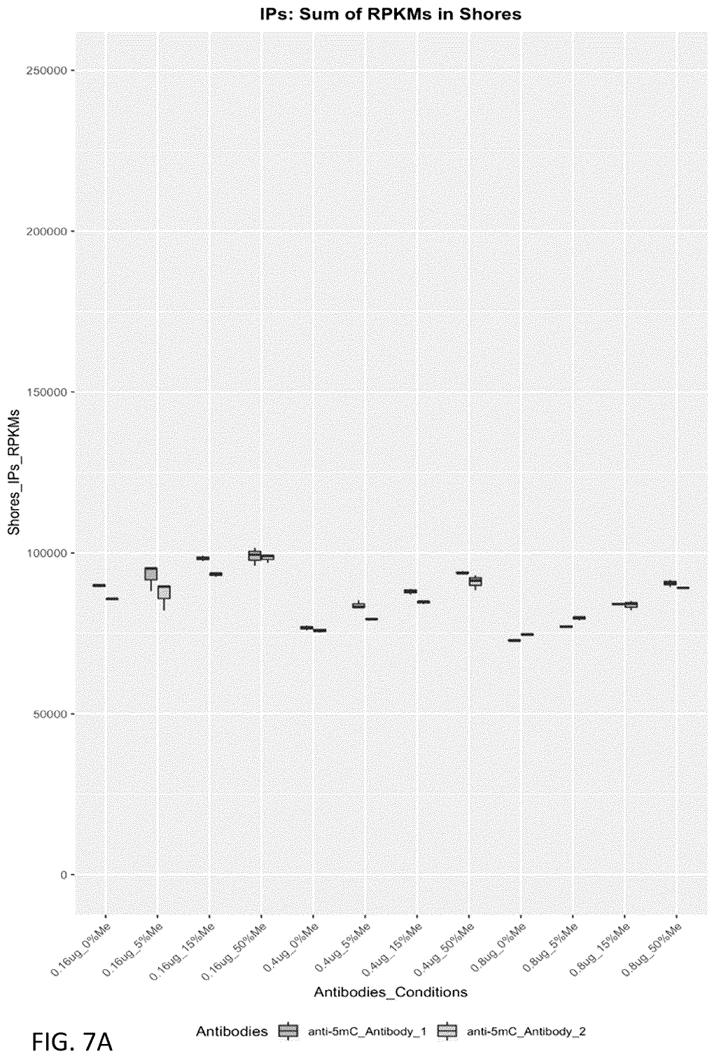

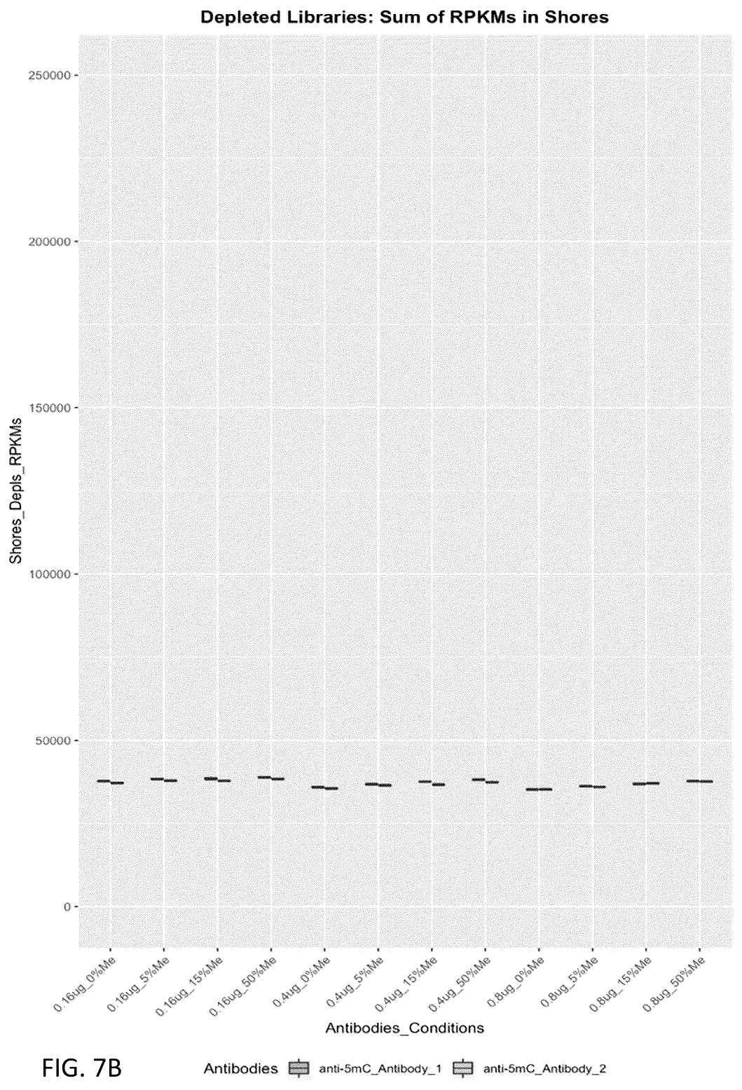

The sum reads per kilobase per million reads (RPKMs) total across all CpG islands in the human genome (human genome build hg38) is shown in FIG. 6A and FIG. 6B for enriched (methylated) and depleted (hypomethylated) libraries, respectively. The sum RPKMs across all CpG island shores in the human genome (human genome build hg38) is shown in FIG. 7A and FIG. 7B for enriched (methylated) and depleted (hypomethylated) libraries, respectively. In each case and for all conditions and tested anti-5mC antibodies, the sums were always observed to be lower for depleted libraries than for enriched libraries.

Thus, it was shown that a strong signal can be obtained for depleted libraries compared to control signals, substantiating the use of depleted libraries to identify the presence of hypomethylated DNA, such as ctDNA, in cfDNA samples.

### Example 6: Calculation of Specificity of cfMeDIP-Seq

This example shows calculation of specificity of cfMeDIP-seq assays using ctDNA samples.

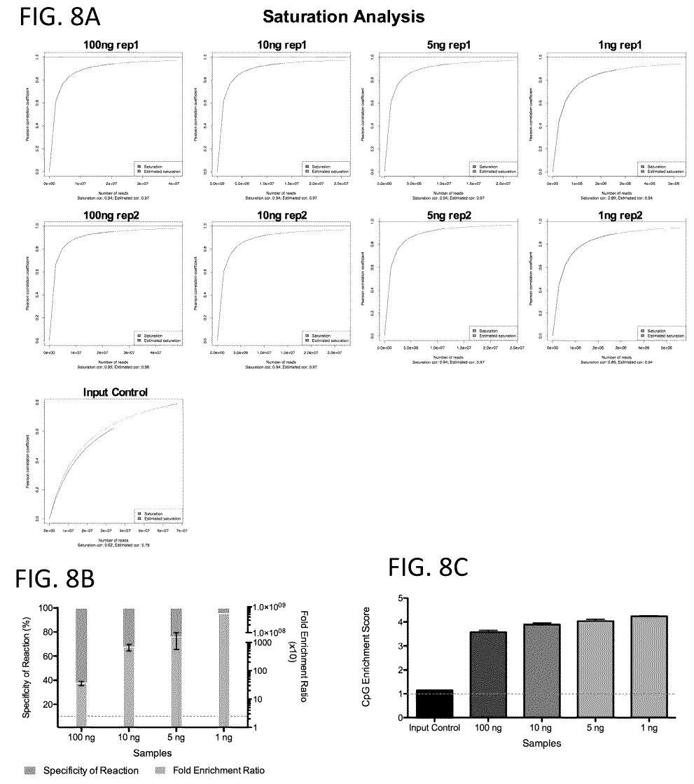

cfMeDIP-seq was validated using DNA from a human colorectal cancer cell line (HCT116), sheared to a fragment size similar to that observed in cfDNA (e.g., as described herein). MeDIP-seq was performed using 100 ng of sheared cell line DNA and using 10 ng, 5 ng, and 1 ng of the same sheared cell line DNA. This was performed in two biological replicates. FIG. 8A shows results of saturation analysis from the Bioconductor package MEDIPS analyzing cfMeDIP-seq data from each replicate for each input concentration from the HCT116 DNA fragmented to mimic plasma cfDNA. The libraries were sequenced to saturation (FIG. 8A) at approximately 30 to 70 million reads per library. The raw reads were aligned to both the human genome and the λ genome, and virtually no alignment was found to the λ genome in the results. Therefore, the addition of the exogenous λ DNA as filler DNA did not interfere with the generation of sequencing data. CpG enrichment score was also calculated as a quality control measure for the immunoprecipitation operation. FIG. 8B shows cfMeDIP-seq results in which four starting DNA concentrations (100, 10, 5, and 1 ng) of HCT116 cell line were assayed in duplicate. Specificity of the reaction was calculated using methylated and unmethylated spiked-in A. thaliana DNA. Fold enrichment ratio was calculated using genomic regions of the fragmented HCT116 DNA, assayed using primers specific for methylated testis (H2B, TSH2B) and unmethylated human DNA region (GAPDH promoter). For all the conditions, more than 99% specificity of the reaction (1-[recovery of spiked-in unmethylated control DNA over recovery of spiked-in methylated control DNA]) was observed, and a very high enrichment of a known methylated region over an unmethylated region (TSH2B and GAPDH, respectively) (FIG. 8B). The horizontal dotted line indicates a fold-enrichment ratio threshold of 25. Error bars represent ±1 s.e.m. FIG. 8C shows CpG enrichment scores indicating that sequenced samples show a robust enrichment of CpGs within the genomic regions from the immunoprecipitated samples compared to the input control. The CpG enrichment score was obtained by dividing the relative frequency of CpGs of the regions by the relative frequency of CpGs in the human genome. Error bars represent ±1 s.e.m. All the libraries showed similar enrichment for CpGs while the input control showed no enrichment, as expected (FIG. 8C), even at extremely low inputs (1 ng).

### Example 7: Calculation of Sensitivity of cfMeDIP-Seq

This example shows calculation of sensitivity of cfMeDIP-seq assays using ctDNA samples.

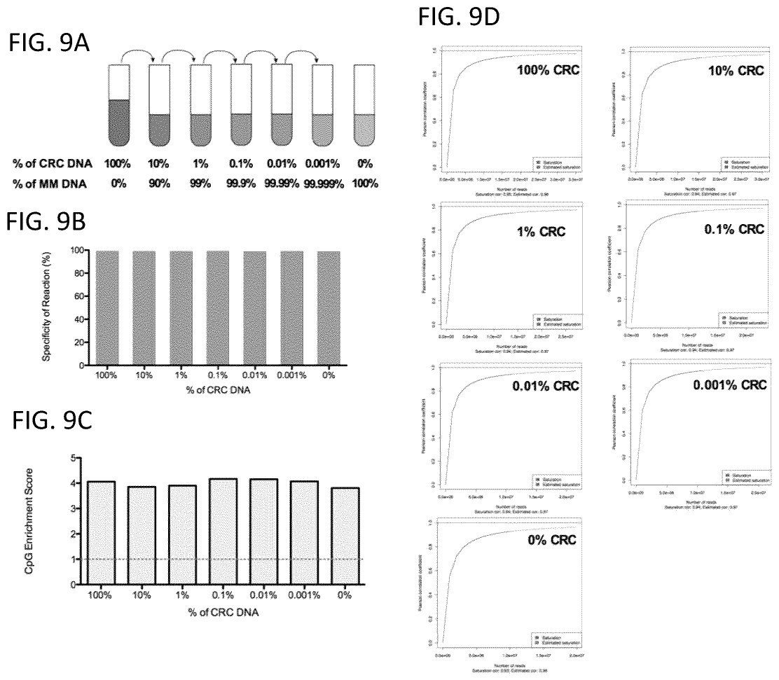

To evaluate the sensitivity of the cfMeDIP-seq protocol, a serial dilution of Colorectal Cancer (CRC) HCT116 cell line DNA into a Multiple Myeloma (MM) MM1.S cell line DNA was performed after shearing each to create mimic cfDNA fragments (FIG. 9A). CRC DNA was diluted from 100%, 10%, 1%, 0.1%, 0.01%, 0.001%, to 0%, and cfMeDIP-seq was performed on each of these dilutions. Ultra-deep (10,000-fold median coverage) targeted sequencing was performed for detection of three point mutations in the same samples. FIG. 9A-FIG. 9D show quality control assays from cfMeDIP-seq using serial dilution, as described herein. FIG. 9A shows a schematic representation of the CRC DNA (HCT116) dilution into MM DNA (MM1.S). FIG. 9B shows specificity of reaction for each dilution, calculated using methylated and unmethylated spiked-in A. thaliana DNA. FIG. 9C shows CpG enrichment scores of the sequenced samples, indicating a strong enrichment of CpGs within the genomic regions from the immunoprecipitated samples. The CpG enrichment score was obtained by dividing the relative frequency of CpGs of the regions by the relative frequency of CpGs in the human genome. FIG. 9D shows saturation analysis results from assays performed with each CRC DNA dilution (100%, 10%, 1%, 0.1%, 0.01%, 0.001%, and 0%). Saturation analysis results were similar in all conditions, indicating excellent sensitivity across a wide range of dilution factors. The observed number of differentially methylated regions identified at each CRC dilution point versus the pure MM DNA using a 5% false discovery rate (FDR) threshold was almost perfectly linear (r²=0.99, p<0.0001) with the expected number of differentially methylated regions based on the dilution factor down to a 0.001% dilution (data not shown). Moreover, the DNA methylation signal within these differentially methylated regions also shows almost perfect linearity (r²=0.99, p<0.0001) between the observed versus expected signal (data not shown). Thus, cfMeDIP-seq displays excellent sensitivity for the detection of cancer-derived DNA, exceeding the performance of variant detection by ultra-deep targeted sequencing using a standard protocol.

### Example 8: Calculation of Percent Recovery Following cfMeDIP-Seq

This example shows calculation of percent recovery of spike-in DNA following cfMeDIP-seq assays.

The success of cfMeDIP-seq experiments was validated through qPCR to detect the presence of the spiked-in A. thaliana DNA, ensuring a percent (%) recovery of unmethylated spiked-in DNA <1% and the percent (%) specificity of the reaction >99% (as calculated by 1-[percent recovery of spiked-in unmethylated control DNA over recovery of spiked-in methylated control DNA]), prior to proceeding to the next step. The optimal number of cycles to amplify each library was determined through the use of qPCR, after which the samples were amplified using the KAPA HiFi Hotstart Mastermix and the NEBNext multiplex oligos added to a final concentration of 0.3 μM. The PCR settings used to amplify the libraries were as follows: activation at 95° C. for 3 min, followed by predetermined cycles of 98° C. for 20 sec, 65° C. for 15 sec and 72° C. for 30 sec and a final extension of 72° C. for 1 min. The amplified libraries were purified using MinElute PCR purification column and then gel size selected with 3% Nusieve GTG agarose gel to remove any adapter dimers. Prior to submission for sequencing, the fold enrichment of a methylated human DNA region (testis-specific H2B, TSH2B) and an unmethylated human DNA region (GAPDH promoter) was determined for the MeDIP-seq and cfMeDIP-seq libraries generated from the HCT116 cell line DNA sheared to mimic cell free DNA (Cell line obtained from ATCC, mycoplasma free). The final libraries were submitted for BioAnalyzer analysis prior to sequencing at the UHN Princess Margaret Genomic Centre on an Illumina HiSeq 2000.

cfMeDIP-seq were performed using different percentages of methylated to unmethylated lambda DNA in the filler component of the protocol as follows:

| | % Methylated | % Unmethylated |
|---|---|---|
| | Filler | Filler |
| Using filler (lambda) DNA | 100 | 0 |
| to increase final amount of | 85 | 15 |
| DNA to 100 ng prior to | 70 | 30 |
| immunoprecipitation, with | 50 | 50 |
| the starting amounts of cell- | 30 | 70 |
| free DNA ranging from | 15 | 85 |
| 10 ng, 5 ng and 1 ng | 0 | 100 |

No Filler DNA used - as is only 10 ng, 5 ng or 1 ng of starting DNA prior to immunoprecipitation

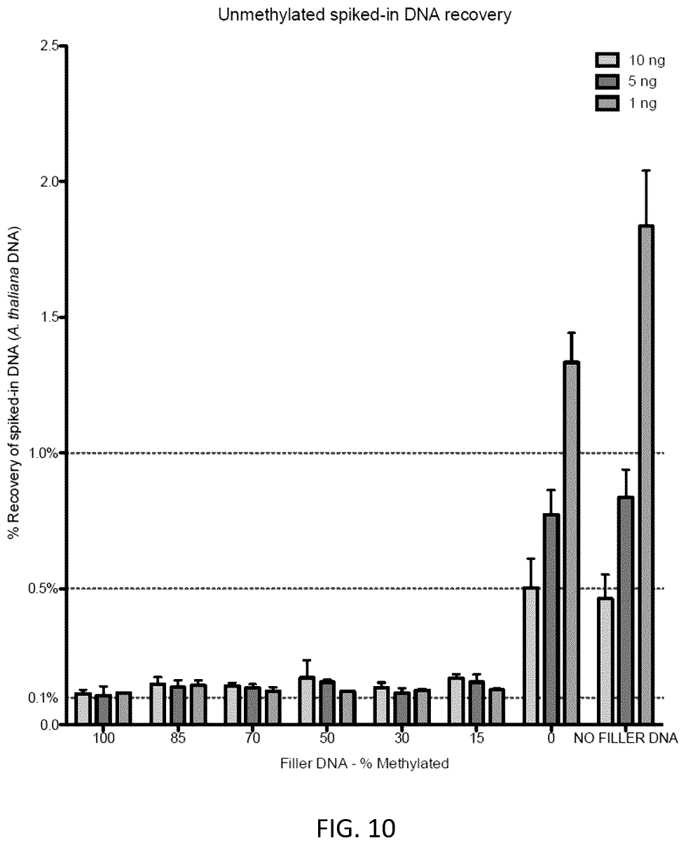

FIG. 10 shows percent (%) recovery of spiked-in unmethylated A. thaliana DNA after cfMeDIP-seq using 10 ng, 5 ng and 1 ng of starting cancer-derived cell-free DNA (ctDNA) amounts (n=3), combined with 90 ng, 95 ng and 99 ng of filler DNA respectively or no filler DNA, prior to immunoprecipitation. The amount of supplemental processed DNA used was varied with respect to the ratio of percent artificially methylated to percent unmethylated lambda supplemental processed DNA present, e.g., to increase final amount prior to immunoprecipitation. The preferred percent recovery of spiked-in unmethylated DNA for these experiments was <1.0%, with lower recovery (e.g., less than 0.5% or 0.1%) resulting in higher percent specificity of reaction.

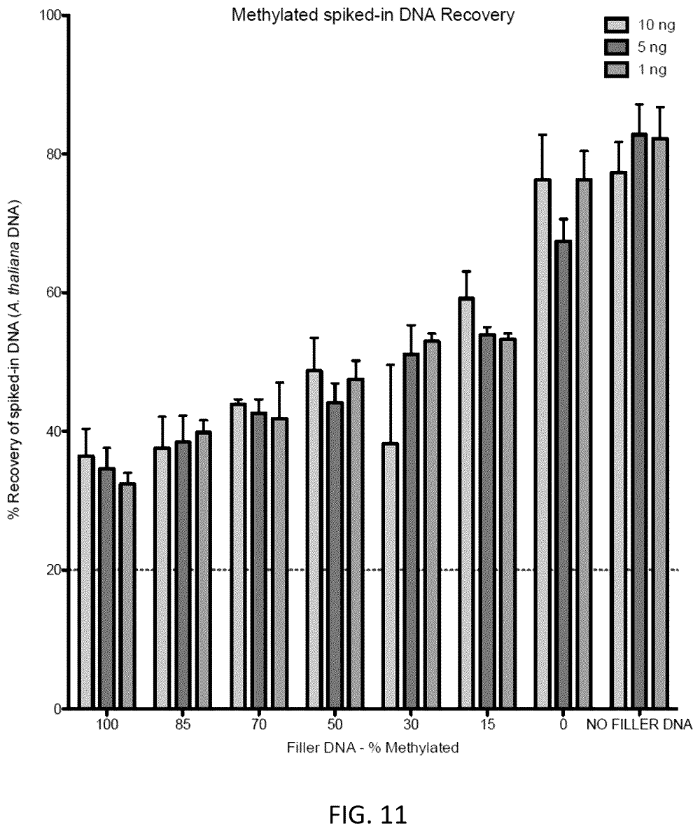

FIG. 11 shows percent (%) recovery of spiked-in methylated A. thaliana DNA after cfMeDIP-seq using 10 ng, 5 ng and 1 ng of starting cancer-derived cell-free DNA (ctDNA) amounts (n=3), combined with 90 ng, 95 ng and 99 ng of filler DNA respectively or no filer DNA, prior to immunoprecipitation. The supplemental processed DNA used was varied with respect to the ratio of percent artificially methylated to percent unmethylated lambda supplemental processed DNA present to increase final amount prior to immunoprecipitation to 100 ng. The target minimum percent recovery of spiked-in methylated DNA in these experiments was 20% or higher.

Supplemental processed DNA (2 DNA) used to increase the final amounts prior to immunoprecipitation to 100 ng, may include artificially methylated DNA in its composition (from 100%-15%), e.g., in order to achieve minimal recovery unmethylated DNA (FIG. 10), while maintaining acceptable yield with respect to recovery of methylated DNA (FIG. 11).

In the samples using 100% unmethylated supplemental processed DNA or no supplemental processed DNA present high percent recovery of unmethylated DNA was observed. These results show that, in some cases, the additional methylated DNA in the supplemental processed DNA can help to occupy the excess antibody present in the reaction, and can minimize the amount of unspecific binding to unmethylated DNA found in the sample. Given that optimizing antibody amounts can be expensive or technically challenging (e.g., in cases where different cell-free DNA samples are used, for example, since the amount of methylated DNA present throughout the sample may be unknown and may differ drastically sample to sample), the supplemental processed DNA can help normalize the different starting amounts and allow for different cell-free DNA samples to be processed in a similar manner (e.g., using same amount of antibody), while still recovering useful methylation data.

### Example 9: Methylated Fraction Fragmentation Analysis

This example shows determination of methylated fraction fragmentation score for nucleic acid populations analyzed as described herein.

A method of using cell-free DNA (cfDNA) fragmentation patterns in methylation fractionated libraries for cancer detection was developed. Methylation fractionated libraries are sequencing libraries enriched for methylated DNA (e.g., immunoprecipitated methylation “enriched” cfMeDIP-seq libraries) or depleted for methylated DNA (e.g., “depleted libraries” as described herein, which can comprise cfMeDIP-seq flowthrough). Uses of this method include identification of the presence of circulating tumor DNA (ctDNA) in a sample of cfDNA obtained from plasma. This method can be used with other sources of cfDNA (e.g., one or more biological samples listed herein, such as urine, CSF, etc.). Briefly, ctDNA was identified by determining occurrence frequencies of short fragments and long fragments in the methylation fractionated libraries. A range of 100-150 bp was used for short fragments and a range of 151-220 bp was used for long fragments; however, it is contemplated that additional or alternate ranges can be used as well. It is contemplated that short fragment length range and long fragment range do not need to be contiguous in MFF analysis. In some cases, a range of from 200 bp to 250 bp, from 150 bp to 200 bp, from 100 bp to 150 bp, from 50 bp to 100 bp, 1 bp to 50 bp, less than 200 bp, or less than 100 bp may be used for identification of short fragment lengths. In some cases, a range of 150 bp to 200 bp, 200 bp to 250 bp, 250 bp to 300 bp, 300 bp to 350 bp, or 350 bp to 400 bp, larger than 200 bp, larger than 300 bp, or larger than 400 bp may be used for identification of long fragment lengths. Regions that are hypomethylated in tumor derived DNA (e.g., ctDNA) can be identified by the presence of an increased frequency of short fragments mapping to that region in the depleted libraries from cancer patients as compared to the depleted libraries of healthy controls. Similarly, regions that are hypermethylated in tumor derived DNA can be identified by the presence of an increased frequency of short fragments mapping to that region in the enriched libraries from cancer patients as compared to the enriched libraries of healthy controls.

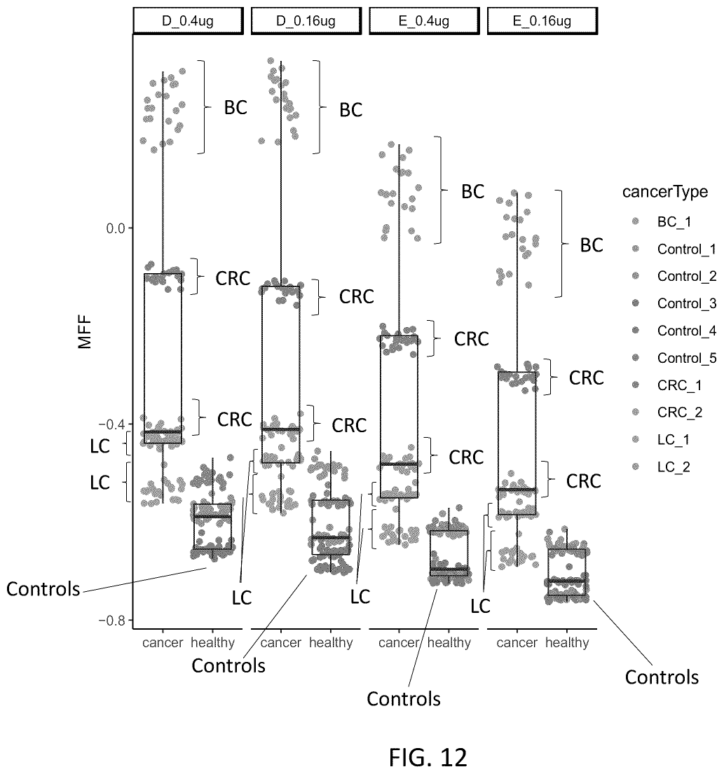

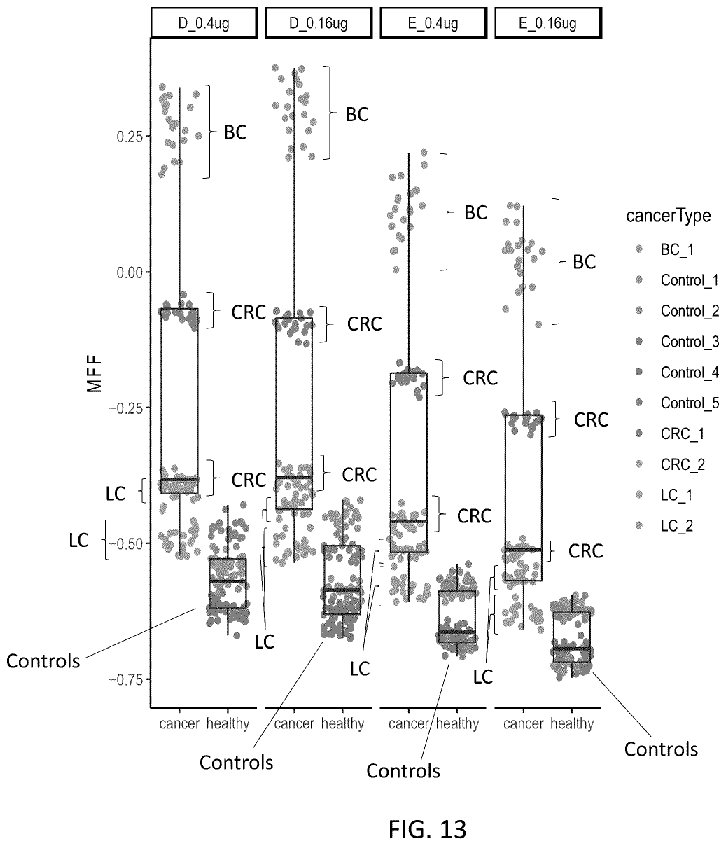

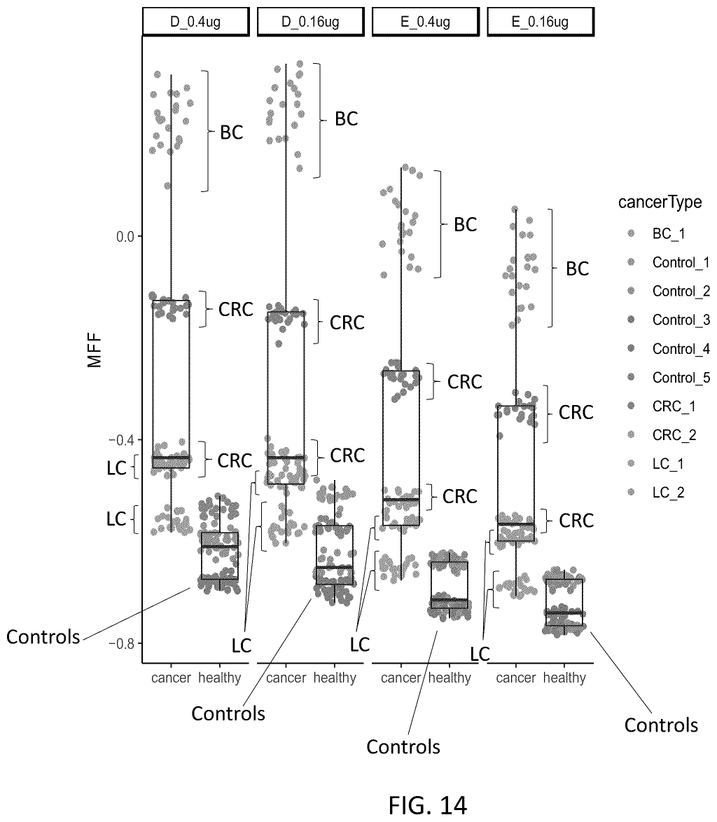

Bioinformatic pipelines were employed that process sequencing libraries generated from the same sample by cfMeDIP-seq. The immunoprecipitated sample was termed “enriched libraries,” as it was enriched for methylated DNA, while the flowthrough (not bound by the 5mC antibody) was termed “depleted libraries,” as it was depleted of methylated DNA. A metric, termed the “Methylation Fractionated Fragmentation” analysis or “MFF” was developed to evaluate the difference in fragmentation profiles between plasma cfDNA obtained from cancer patients (n=5) and healthy donors (n=5) in the methylation depleted and methylation enriched libraries. FIG. 12 shows boxplots of genome-wide MFF score distributions from cancer patients or healthy control samples. For each sample listed in the legend of FIG. 12 (e.g., listing analyzed cancer types (“cancerType”); BC: breast cancer; Control: healthy; CRC: colorectal cancer; LC: lung cancer), an MFF score value was calculated for each chromosome (1 to 22). Results obtained with this analysis method showed that plasma cfDNA from cancer patients (left side bar for each condition in FIG. 12) had a higher fraction of shorter fragments, as measured by MFF score, as compared to healthy individuals (right side bar for each condition in FIG. 12) in both the enriched libraries (“E_0.4 μg”, right-center pair of bars corresponding to 0.4 micrograms (μg) of anti-5mC antibody used, and “E_0.16 μg,” rightmost pair of bars corresponding to 0.16 μg of anti-5mC antibody used) and depleted libraries (“D_0.4 μg,” leftmost pair of bars corresponding to 0.4 micrograms (μg) of anti-5mC antibody used, and “D_0.16 μg,” left-center pair of bars corresponding to 0.16 μg of anti-5mC antibody used) (FIG. 12). Even at a significantly lower sequencing depth (with enriched libraries sequenced to an average of 47 million paired reads per sample and depleted libraries sequenced to an average of 10.8 million paired reads per sample), the depleted libraries showed a better separation between cancer patients and healthy controls, due to the global DNA hypomethylation that occurs in cancer DNA (FIG. 12). Similar to using the genome-wide approach described above, calculating the MFF score for only regions that overlap with CpG shores (FIG. 13) or long terminal repeats (LTRs) (FIG. 14), which are features frequently hypomethylated in cancer, showed an increase in MFF scores was observed in cancer patients compared to controls in both enriched and depleted libraries (FIG. 13, FIG. 14). Again, depleted libraries showed the best separation between cancer and controls (FIG. 13, FIG. 14). For each sample listed in the legends of FIG. 13 and FIG. 14 (e.g., listing analyzed cancer types (“cancerType”); BC: breast cancer; Control: healthy; CRC: colorectal cancer; LC: lung cancer), an MFF score value was calculated for each chromosome (1 to 22). This same approach (e.g., comprising MFF analysis) can also be used for other genomic features (e.g., CpG shores, Open Sea, LINE1 retroelements, SINEs, etc.), in addition to LTRs.

Finally, the MFF scores can be used to identify genomic regions of interest that have a differential MFF score between cancer and controls in the depleted or enriched libraries (FIG. 15-FIG. 19). Again, the MFF scores from the depleted libraries provided the best discrimination between cancer versus controls. For this example, five 5 Mb bins to identify genomic regions of interest were used here; however, bins of other sizes (e.g., less than 5 Mb, greater than 5 Mb, a bin from 1 Mb to 5 Mb, a bin from 5 Mb to 10 Mb, a bin less than 1 Mb, or a bin greater than 10 Mb) can be used.

In summary, these data show that this technology is capable of detecting cancer-specific fragmentation patterns at methylated and unmethylated cfDNA fractions and that populations of nucleic acids (and/or biological samples from which they are derived) from subjects having cancer and control (e.g., healthy) subjects can be distinguished using MFF score analysis. The MFF scores from the depleted libraries performed the best even at shallow sequencing. This suggests that MFF analysis is a cost-efficient method for ctDNA detection. It is contemplated that improved sensitivity of ctDNA detection by cfMeDIP-seq can be obtained by expanding the repertoire of sequenced ctDNA fragments (i.e., methylated and unmethylated) for detection and subsequent analysis.

Method operations used for cfMeDIP-seq with MFF results shown in FIGS. 12-19 were as follows. 10 ng of cancer patient or healthy donor cfDNA was utilized with 0.1 ng of Adela spike-in control DNA, carried out in duplicates. The DNA was subjected to library preparation using the Kapa Hyper Prep Kit in combination with the IDT xGen CS Adapter (IDT, Cat #1080799), following manufacturer's protocol with minor modifications. In brief, after end-repair and A-tailing, 0.327 μM of xGen CS adapter was ligated to the DNA following an incubation of 30 mins at 20° C. After purification of the adapter ligated DNA using AMPure XP beads, 5% of the DNA was saved as the input control. The remaining DNA was combined with A filler DNA to increase starting DNA input to 100 ng prior to MeDIP. MeDIP was carried out as previously published (Shen, S. Y., Burgener, J. M., Bratman, S. V., & De Carvalho, D. D. (2019) “Preparation of cfMeDIP-seq libraries for methylome profiling of plasma cell-free DNA.” Nature protocols, 14 (10), 2749-2780), which is incorporated herein by reference for all purposes, including cfMeDIP-seq method operations, with some modifications). For each patient sample, one replicate sample was subjected to MeDIP-seq using 0.16 μg of 5-mC antibody and the other was subjected to MeDIP-seq using 0.4 μg of 5-mC antibody. In each reaction, after the antibody incubation, the remaining supernatant known as the depleted library, was purified using Zymo RNA Clean & Concentrator™-5 kit. The cfMeDIP-seq libraries were purified using the previously published protocol, followed by indexing and amplification using 15 cycles of PCR using IDT xGen UDI primers (IDT, Cat #10005922). The purified depleted libraries were indexed and amplified using 7 cycles of PCR using the same PCR mastermix and protocol. The previously saved input control DNA for each respective sample was also amplified using the same PCR mastermix and protocol used for MeDIP, reducing the PCR cycle number to 10 cycles. All final libraries were purified using AMPure XP beads.

All generated libraries, cfMeDIP-seq, depleted and input control libraries were sequenced on the NovaSeq 6000 with configuration of paired-end 100 bp.

Calculation of the Methylated Fractionated Fragmentation (MFF) score was performed as follows. The long fragment fraction (LFF) was subtracted from the short fragment fraction (SFF). To calculate the SFF or LFF, the number of fragments between 100-150 bp or 151-220 bp were divided by the number of fragments between 100-220 bp respectively. The calculation was performed for each binned region of the genome. Let s and l denote the number of fragments between 100-150 bp and 151-220 bp respectively. Let k denote an individual binned region of interest. This gives

$${SFF}_{k} = \frac{{s}_{k}}{{s}_{k} + {l}_{k}}$$

$${LFF}_{k} = \frac{{l}_{k}}{{s}_{k} + {l}_{k}}$$

$${MFF}_{k} = {SFF}_{k} - {LFF}_{k}$$

All cfMeDIP-seq (“enriched libraries”) and depleted libraries were put through the pipeline which performs standard bioinformatics operations including trimming of raw reads in FASTQ files, aligning them to the human genome build hg38 to generate BAM files which are subsequently converted to BED file format which provides the chromosome, start, and end site location of each mapped read.

The fragment length of reads within each BED file was extracted, selecting fragments that overlapped with the background file and any additional regions of interest. Fragment counts were summarized across chromosome 1 to 22 between 100-150 bp and 151-220 bp, designated as short and long fragment respectively. From these count matrices, the MFF value was calculated.

To evaluate the initial performance of the MFF metric, the distribution of MFF values per chromosome was calculated for each cancer patient sample and each healthy donor sample. Limiting analysis to regions within the background file, the distribution of cancer patient samples was compared to healthy donors, for cfMeDIP-seq and depleted libraries using 0.16 micrograms (μg) or 0.4 μg of anti-5mC antibody. It was observed that depleted libraries produced using 0.4 μg or 0.16 μg of anti-5mC antibody demonstrated increased MFF values across cancer samples and healthy donors compared to enriched libraries, as shown in FIG. 12. This trend was consistent when analysis was limited to non-CpG islands (shown here is analysis for CpG Shore regions) as shown in FIG. 13, as well as when analysis was limited to repeat regions (shown here are long terminal repeat regions (LTRs)) as shown in FIG. 14.

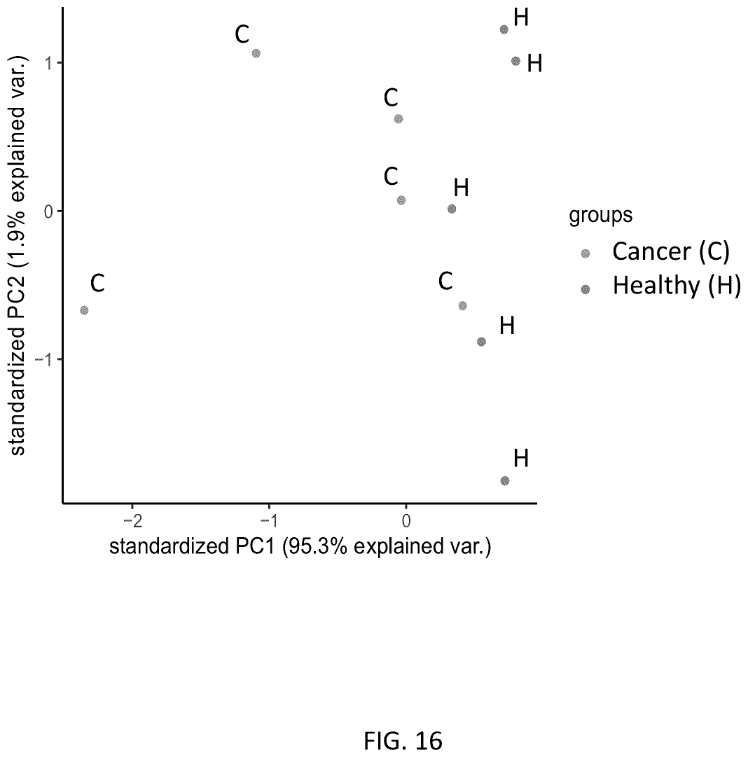

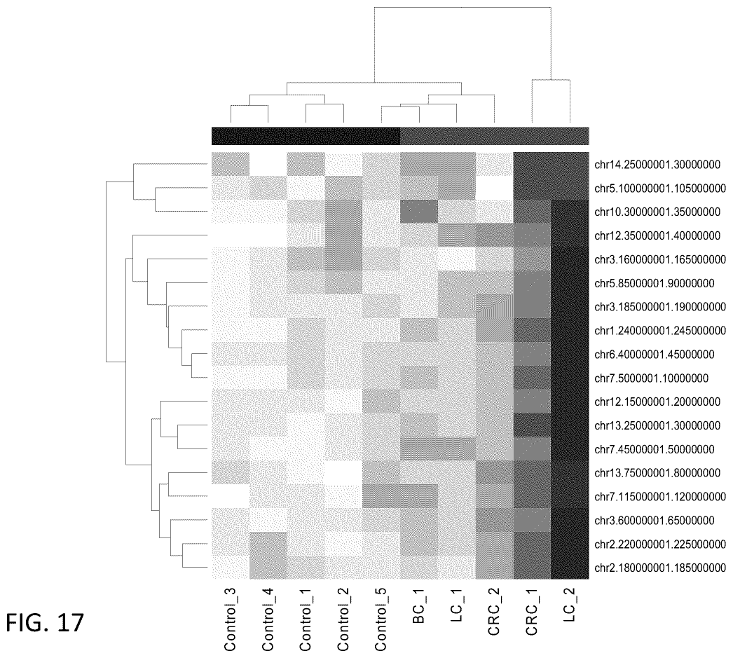

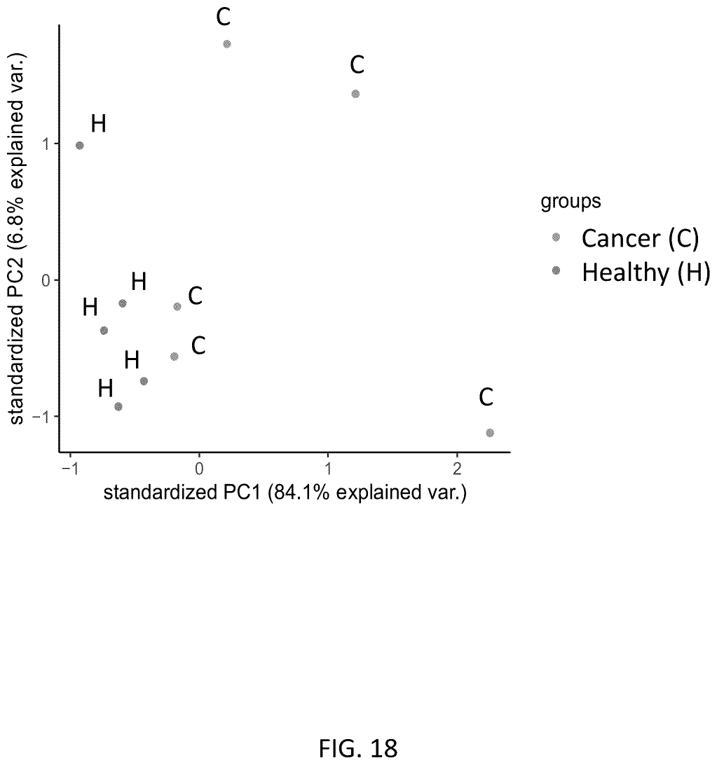

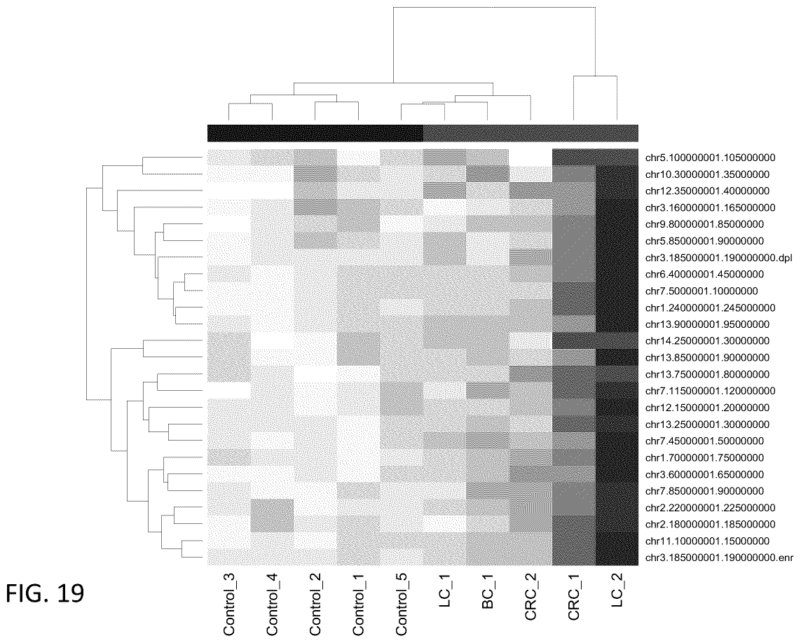

Counts across five megabase (5 Mb) regions (e.g., instead of across chromosomes) were then summarized to confirm that MFFs with elevated values in cancer samples versus healthy donors could be stratified. First, the performance of elevated MFFs from enriched libraries was evaluated, across all enriched libraries (FIG. 15, FIG. 16). Heatmap analysis of enriched MFFs of interest, across all enriched (0.16 μg of 5mC antibody) MFF libraries, is shown in FIG. 15. PCA analysis of enriched MFFs of interest, across all enriched (0.16 μg of 5mC antibody) MFF libraries, is shown in FIG. 16. This analysis was then repeated for elevated MFFs from depleted libraries, across depleted libraries from 0.4 microgram (μg) anti-5mC antibody (FIG. 17, FIG. 18). Heatmap analysis of depleted MFFs of interest, across all depleted (0.4 μg of 5mC antibody) MFF libraries, is shown in FIG. 17. PCA analysis of depleted MFFs of interest, across all depleted (0.4 μg of 5mC antibody) MFF libraries, is shown in FIG. 18. Finally, the combined performance of elevated MFFs from enriched libraries as well as elevated MFFs from depleted libraries were evaluated. FIG. 19 shows heatmap analysis of depleted MFFs of interest across all depleted (0.4 μg of 5mC antibody) MFF libraries and enriched MFFs of interest across all enriched (0.16 μg of anti-5mC) MFF libraries. Overlapping regions of interest between depleted and enriched MFF libraries are denoted in FIG. 19 by “dpl” and “enr” respectively.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

1.-52. (canceled)

53. A method for nucleic acid processing, comprising:
(a) providing a mixture comprising (i) a first plurality of nucleic acid molecules of a nucleic acid sample of a subject and (ii) a second plurality of nucleic acid molecules that is not from said subject;
(b) with aid of said second plurality of nucleic acid molecules, depleting said mixture of one or more nucleic acid molecules of said first plurality of nucleic acid molecules that are hypermethylated, thereby yielding a remainder of said first plurality of nucleic acid molecules that is unmethylated or hypomethylated relative to said one or more nucleic acid molecules; and
(c) identifying a sequence of said remainder of said first plurality of nucleic acid molecules or derivatives thereof.

54. The method of claim 53, further comprising contacting said mixture with a binder selective for methylated regions of nucleic acid molecules under a sufficient condition for said binder to bind said methylated regions of nucleic acid molecules.

55. The method of claim 53, wherein said first plurality of nucleic acid molecules comprises deoxyribonucleic acid (DNA) molecules.

56. The method of claim 53, wherein said nucleic acid sample is a cell-free DNA (cfDNA) sample.

57. The method of claim 53, wherein said second plurality of nucleic acid molecules comprises deoxyribonucleic acid (DNA) molecules.

58. The method of claim 53, wherein said second plurality of nucleic acid molecules does not align to a human genome.

59. The method of claim 53, wherein said second plurality of nucleic acid molecules comprises a fragment length of about 50 basepairs (bp) to about 800 bp.

60. The method claim 53, wherein said remainder of said first plurality of nucleic acid molecules comprises a fragment length of at least about 300 bp.

61. The method of claim 53, wherein said remainder of said first plurality of nucleic acid molecules is substantially deprived of CpG genomic islands.

62. The method of claim 53, wherein said remainder of said first plurality of nucleic acid molecules comprises long interspersed nuclear elements (LINEs), short interspersed nuclear elements (SINEs), or long terminal repeat (LTR) elements.

63. The method of claim 53, wherein said remainder of said first plurality of nucleic acid molecules comprises CpG shores.

64. The method of claim 54, wherein said binder is selected from the group consisting of an anti-5-methylcytosine antibody or a derivative thereof, an anti-5-carboxylcytosine antibody or a derivative thereof, an anti-5-formylcytosine antibody or a derivative thereof, an anti-5-hydroxymethylcytosine antibody or a derivative thereof, an anti-3-methylcytosine antibody or a derivative thereof, and any combinations thereof.

65. The method of claim 53, further comprising (d) purifying said remainder of said first plurality of nucleic acid molecules to yield a plurality of purified nucleic acid molecules.

66. The method of claim 65, further comprising amplifying said plurality of purified nucleic acid molecules.

67. The method of claim 66, further comprising subjecting amplified nucleic acid molecules or derivative thereof to sequencing.

68. The method of claim 67, wherein said sequencing is performed at a low sequencing depth.

69. The method of claim 67, wherein said sequencing is performed at a sequencing depth of from 0.1× to 10×.

70. The method of claim 53, further comprising using an array or polymerase chain reaction (PCR) to identify said sequence of said remainder of said first plurality of nucleic acid molecules or derivatives thereof.

71. The method of claim 53, wherein said remainder of said first plurality of nucleic acid molecules comprises a sum of Reads Per Kilobase per Million reads (RPKMs) that is lower than 50,000 across a plurality of CpG islands.

72. The method of claim 53, wherein said remainder of said first plurality of nucleic acid molecules comprises a CpG enrichment score that is lower than 2.

73. The method of claim 53, wherein the method further comprises (d) computer processing said sequence of said remainder of said first plurality of nucleic acid molecules or derivatives thereof to obtain a fragment length profile of said subject.

74. The method of claim 73, wherein said fragment length profile is used to generate a fragment fraction score.

75. The method of claim 74, wherein said fragment fraction score indicates whether said subject has or is at an increased risk of having a cancer.
